(19) United States
(12) Patent Application Publication
(10) Pub. No.: US 2008/0242618 A1
(43) Pub. Date: Oct. 2, 2008

(54) Stratification

(76) Inventors: Nisar A. Khan, Rotterdam (NL); Gert Wensvoort, Koekange (NL); Robert Benner, Barendrecht (NL); Leonie A. Boven, Rotterdam (NL)

Correspondence Address: TRASK BRITT, P.O. BOX 2550, SALT LAKE CITY, UT, 84110, US

(21) Appl. No.: 11/982,293

(22) Filed: Oct. 31, 2007

Related U.S. Application Data
(63) Continuation-in-part of application No. 10/409,657, filed on Apr. 8, 2003.
Application No. 10/409,657 is a continuation-in-part of application No. 10/028,075, filed on Dec. 21, 2001.
Application No. 10/028,075 is a continuation-in-part of application No. 11/346,761, filed on Feb. 3, 2006.
Application No. 11/346,761 is a continuation of application No. 11/286,571, filed on Nov. 23, 2005, now abandoned.
Application No. 11/286,571 is a continuation-in-part of application No. 10/409,654, filed on Apr. 8, 2003.
Application No. 10/409,654 is a continuation-in-part of application No. 10/028,075, filed on Dec. 21, 2001.

(51) Int. Cl. A61K 38/08; A61K 38/06; A61K 38/07

(52) U.S. Cl. 514/16; 514/18; 514/17

(57) ABSTRACT

Provided is the use of a gene-regulatory peptide or functional analogue thereof for the treatment of a disease comprising an inflammatory condition and/or a counter-inflammatory condition wherein a subject suffering from the disease is subjected to a diagnostic process aimed at determining inflammatory disease stage of the subject and where treatment is selected depending on the outcome of the determination of disease stage.

### CROSS-REFERENCE TO RELATED APPLICATIONS

This application is a continuation-in-part of co-pending patent application U.S. Ser. No. 10/409,657, filed Apr. 8, 2003, now U.S. Pat. No. ______, which is a continuation-in-part of U.S. Ser. No. 10/028,075, filed Dec. 21, 2001, now U.S. Pat. No. ______, and of U.S. Ser. No. 11/346,761, filed Feb. 3, 2006, now U.S. Pat. No. ______, which is a continuation of U.S. Ser. No. 11/286,571, filed Nov. 23, 2005, now U.S. Pat. No. ______, which is a continuation-in-part of U.S. Ser. No. 10/409,654, filed Apr. 8, 2003, now U.S. Pat. No. ______, which is a continuation-in-part of U.S. Ser. No. 10/028,075, filed Dec. 21, 2001, the content of the entirety of each of which are hereby incorporated herein by this reference.

### TECHNICAL FIELD

The current invention relates to biotechnology generally, and the body's innate way of modulation of important physiological processes and builds on insights reported in WO 99/59717, WO 01/00259 and PCT/NL/00639, the content of the entirety of each of which are hereby incorporated herein by this reference.

### BACKGROUND

In these earlier patent applications, small gene-regulatory peptides are described that are present naturally in pregnant women and are derived from proteolytic breakdown of placental gonadotropins such as human chorionic gonadotropin (hCG) produced during pregnancy. These peptides (in their active state often only at about four to six amino acids long) were shown to have unsurpassed immunological activity that they exert by regulating expression of genes encoding inflammatory mediators such as cytokines. Surprisingly, it was found that breakdown of hCG provides a cascade of peptides that help maintain a pregnant woman's immunological homeostasis. These peptides are nature's own substances that balance the immune system to assure that the mother stays immunologically sound while her fetus does not get prematurely rejected during pregnancy but instead is safely carried through its time of birth.

Where it was generally thought that the smallest breakdown products of proteins have no specific biological function on their own (except possibly to serve as antigen for the immune system), it now emerges that the body in fact routinely utilizes the normal process of proteolytic breakdown of the proteins it produces to generate important gene-regulatory compounds, short peptides that control the expression of the body's own genes. Apparently, the body uses a gene-control system ruled by small broken down products of the exact proteins that are encoded by its own genes.

During pregnancy, the maternal system introduces a status of temporary immuno-modulation which results in suppression of maternal rejection responses directed against the fetus. Paradoxically, during pregnancy, often the mother's resistance to infection is increased and she is found to be better protected against the clinical symptoms of various auto-immune diseases such as rheumatism and multiple sclerosis. The protection of the fetus can thus not be interpreted only as a result of immune suppression. Each of the above three applications have provided insights by which the immunological balance between protection of the mother and protection of the fetus can be understood.

Inventors hereof have shown that certain short breakdown products of hCG (i.e., short peptides which can easily be synthesized, if needed modified, and used as pharmaceutical composition) exert a major regulatory activity on pro- or anti-inflammatory cytokine cascades that are governed by a family of crucial transcription factors, the NFkappaB family which stands central in regulating the expression of genes that shape the body's immune response.

Most of the hCG produced during pregnancy is produced by cells of the placenta, the exact organ where cells and tissues of mother and child most intensely meet and where immuno-modulation is most needed to fight off rejection. Being produced locally, the gene-regulatory peptides which are broken down from hCG in the placenta immediately balance the pro- or anti-inflammatory cytokine cascades found in the no-mans land between mother and child. Being produced by the typical placental cell, the trophoblast, the peptides traverse extracellular space; enter cells of the immune system and exert their immuno-modulatory activity by modulating NFkappaB-mediated expression of cytokine genes, thereby keeping the immunological responses in the placenta at bay.

### SUMMARY OF THE INVENTION

It is herein postulated that the beneficial effects seen on the occurrence and severity of auto-immune disease in the pregnant woman result from an overspill of the hCG-derived peptides into the body as a whole; however, these effects must not be overestimated, as it is easily understood that the further away from the placenta, the less immuno-modulatory activity aimed at preventing rejection of the fetus will be seen, if only because of a dilution of the placenta-produced peptides throughout the body as a whole. However, the immuno-modulatory and gene-regulatory activity of the peptides should by no means only be thought to occur during pregnancy and in the placenta; man and women alike produce hCG, for example in their pituitaries, and nature certainly utilizes the gene-regulatory activities of peptides in a larger whole.

Consequently, a novel therapeutic inroad is provided, using the pharmaceutical potential of gene-regulatory peptides and derivatives thereof. Indeed, evidence of specific up- or down-regulation of NFkappaB driven pro- or anti-inflammatory cytokine cascades that are each, and in concert, directing the body's immune response was found in silico in gene-arrays by expression profiling studies, in vitro after treatment of immune cells and in vivo in experimental animals treated with gene-regulatory peptides. Also, considering that NFkappaB is a primary effector of disease (A. S. Baldwin, J. Clin. Invest., 2001, 107:3-6), using the hCG derived gene-regulatory peptides offer significant potential for the treatment of a variety of human and animal diseases, thereby tapping the pharmaceutical potential of the exact substances that help balance the mother's immune system such that her pregnancy is safely maintained.

### BRIEF DESCRIPTION OF THE DRAWINGS

FIG. 1: Hemorrhagic Shock model (HS) (*=time of administration peptide A, B or C in the peptide groups).

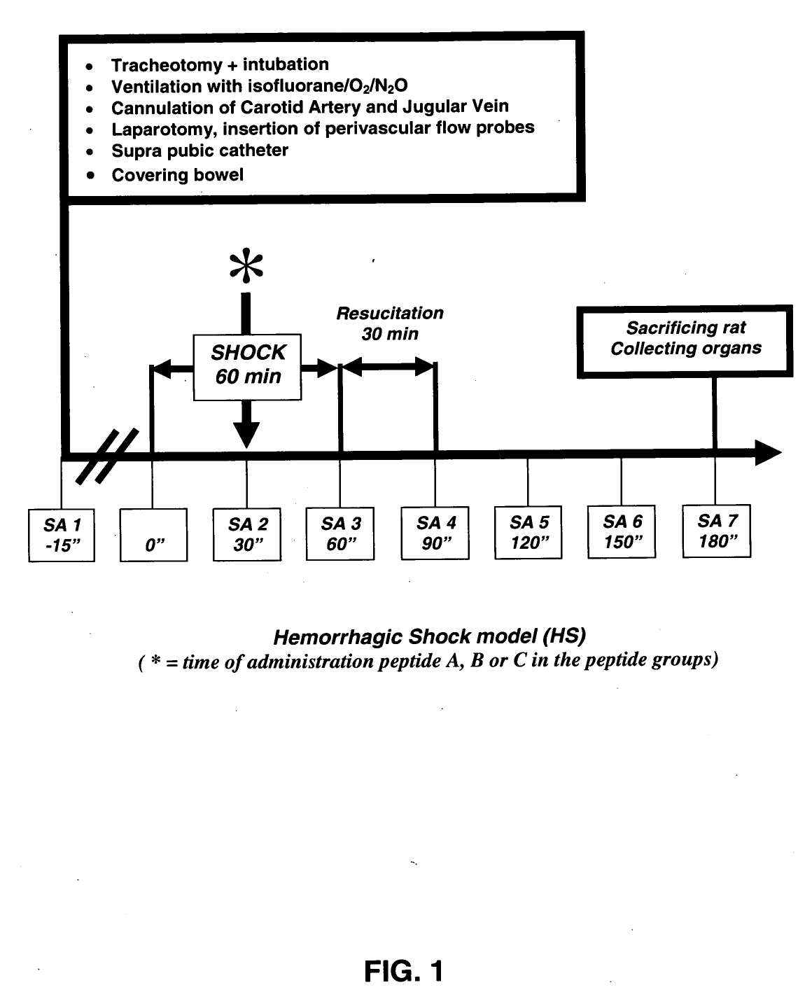

FIG. 2: Mean Arterial Pressure in sham, shock, and Peptide A, B and C experiments.

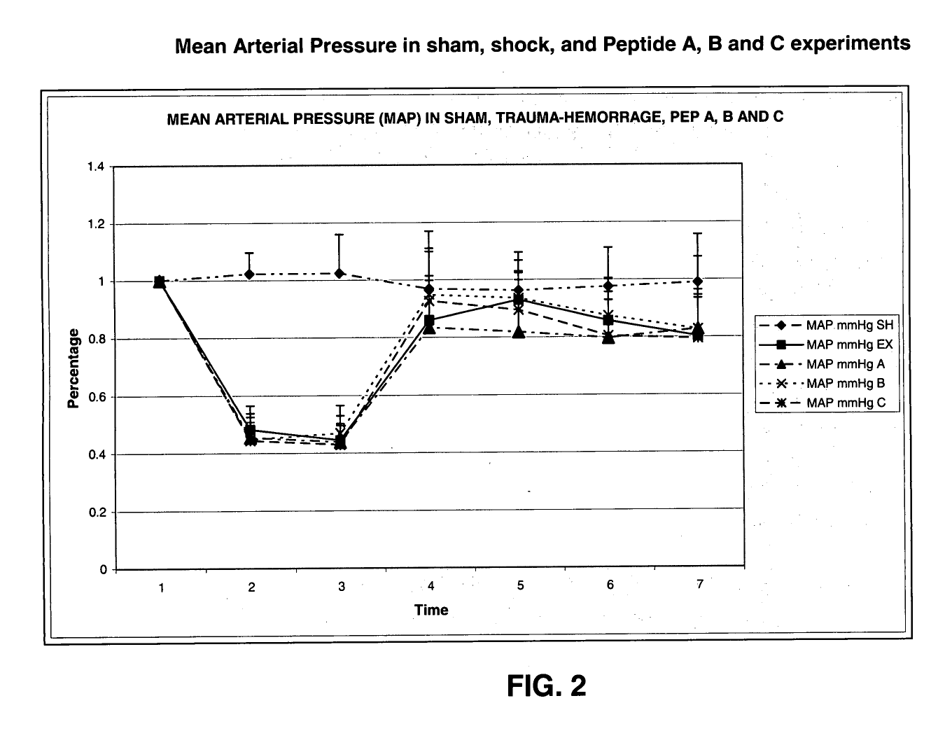

FIG. 3: Hematocrit in (from left to right) sham, shock, and Peptide A, B and C experiments.

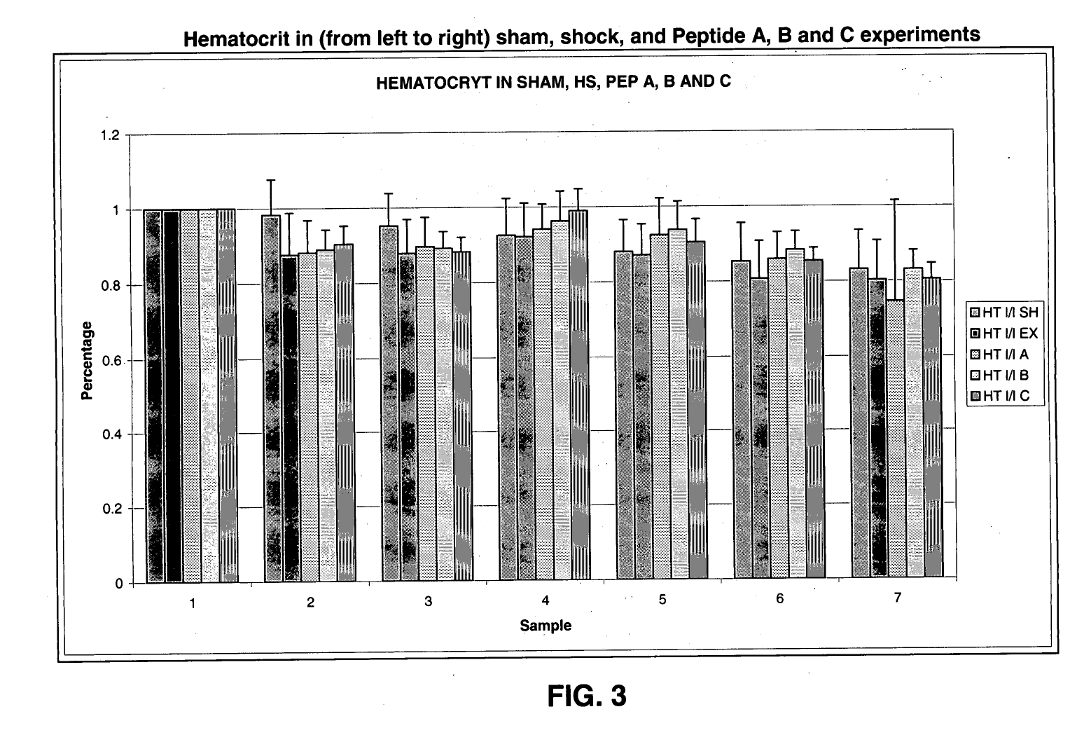

FIG. 4: Leukocytes during sham, trauma-hemorrhage, pep A, B and C experiments.

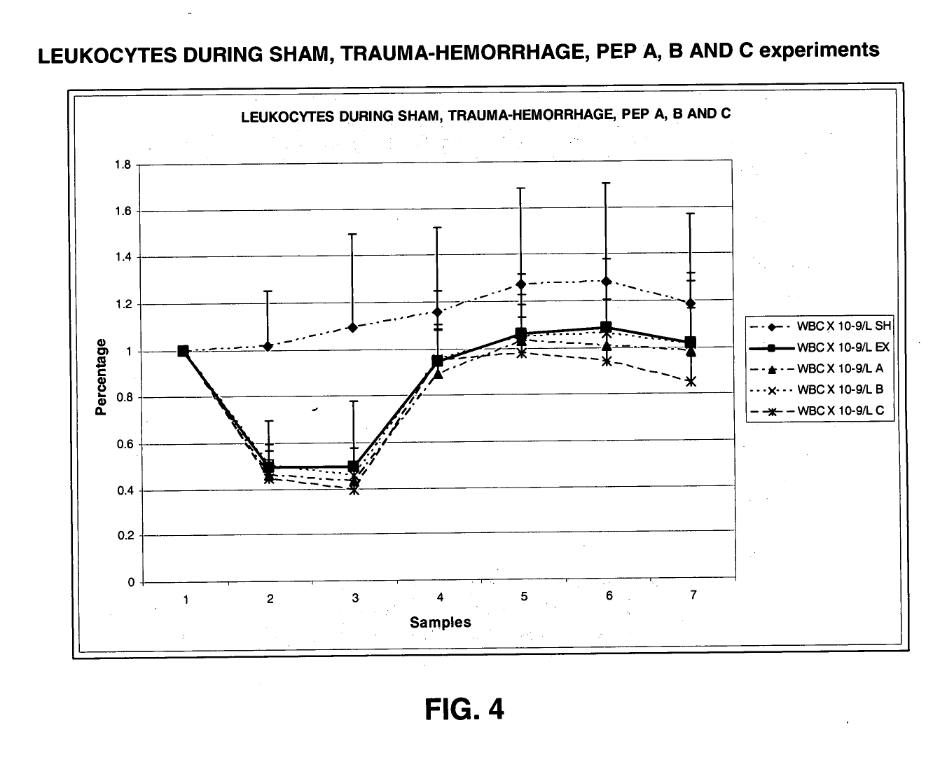

FIG. 5: Macrophages (MO) and granulocytes (GR) in (from left to right) sham, trauma-hemorrhagic shock, and Peptide A, B and C experiments.

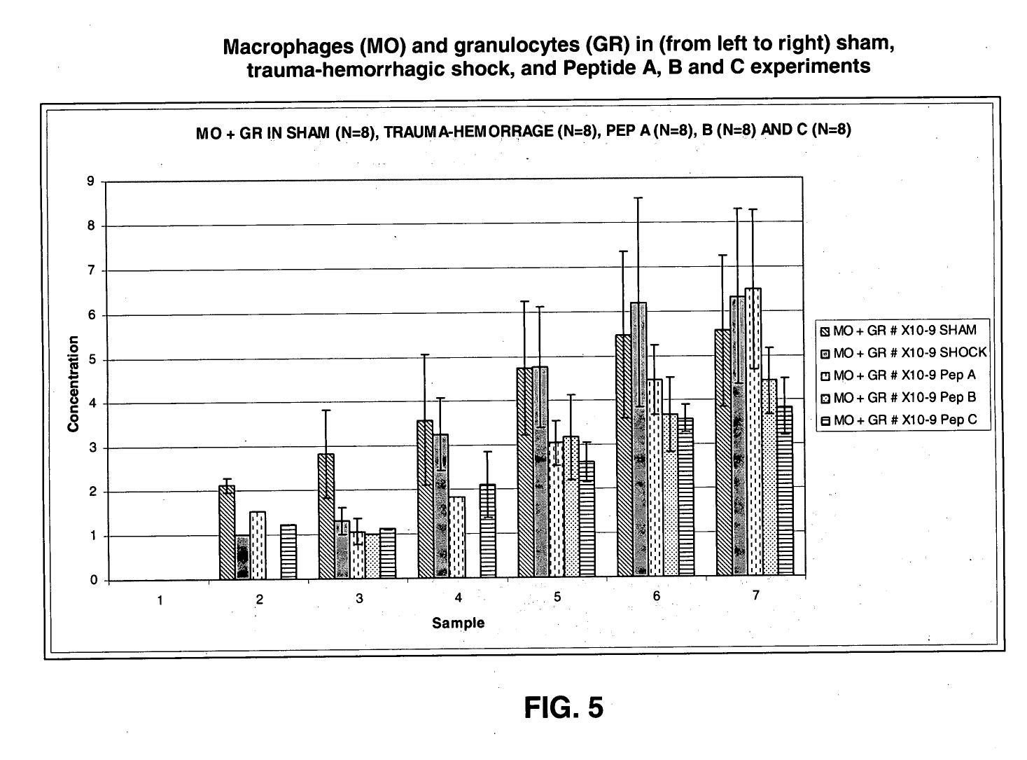

FIG. 6: Arterial blood flow in (from left to right) sham, shock, and Peptide A, B and C experiments.

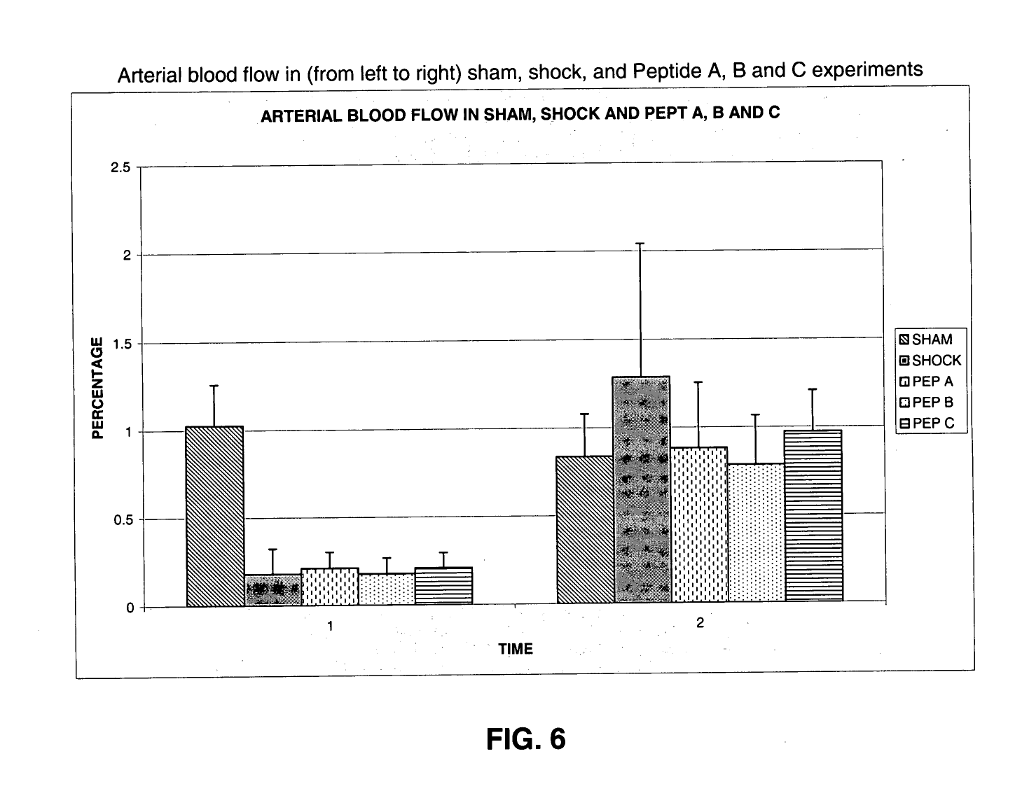

FIG. 7: Hemorrhagic shock model. A) Schematic representation of the experimental design. B) The measured mmHg was recalculated in percentages to standardize the experiment and to compensate for animal differences. C) Percentage of leukocytes in blood during various time points of the experiment.

FIG. 8: TNF-α plasma levels in different experimental groups determined at 15 minutes before and 30, 60, 90, 120, 150 and 180 minutes after the onset of hemorrhagic shock. □ Sham, O HS, ∇ HS/LQGV (SEQ ID NO:1), ⋄ HS/AQGV (SEQ ID NO:2), Δ HS/LAGV (SEQ ID NO:10). Each figure represents one animal.

FIG. 9: IL-6 plasma levels in different experimental groups determined at 120, 150 and 180 minutes after the onset of hemorrhagic shock □ Sham, O HS, ∇ HS/LQGV (SEQ ID NO:1), ⋄ HS/AQGV (SEQ ID NO:2), Δ HS/LAGV (SEQ ID NO:10). Each figure represents one animal.

FIG. 10: Transcript levels for [symbol] ) TNF-α, [symbol] ) IL-6 and [symbol] ) ICAM-1 in the liver, 180 minutes after the onset of hemorrhagic shock. Data expressed are correlated to GAPDH expression. □ Sham, O HS, ∇ HS/LQGV (SEQ ID NO:1), ⋄ HS/AQGV (SEQ ID NO:2), Δ HS/LAGV (SEQ ID NO:10). Each figure represents one animal.

FIGS. 11A and 11B: Depict the same experiment but reflect the scores of two independent observers RK and JV. Peptide A=LAGV; Peptide B=AQGV; Peptide G=VLPALPQ; Peptide I=LQGV. Treatment protocol: Daily application of 4% imiquimod cream (day 0-day 6) on shaved back; 300 μg/mouse peptide in PBS i.p. on days −1, 1, 3 and 5. Scoring for redness, scaling and skin thickness, daily, blindly. Cumulative score=redness+scaling+thickness (scale 0-12).

FIG. 12: Peptide I=LQGV. Treatment protocol: Daily application of 5% imiquimod cream (day 1-day 5) on shaved back and ear. Immediately before imiquimod application, treat skin from back and ear with petroleum ether to remove fat and scales (also groups not treated with petroleum ether); 500 μg/mouse peptide I in PBS i.p. on days 1, 3 and 5. Measuring ear thickness on days 1, 3, and 5. Scoring for redness, scaling and skin thickness, daily, blindly. Cumulative score=redness+scaling+thickness (scale 0-12).

### DETAILED DESCRIPTION OF THE INVENTION

This disclosure relates in particular to methods of treating disease comprising an inflammatory condition and/or a counter-inflammatory condition.

Inflammation is a general name for reactions occurring after most kinds of tissue injuries or infections or immunologic stimulation as a defense against foreign or altered endogenous substances. Inflammatory reactions involve a number of biochemical and cellular alterations the extent of which correlates with the extent of the initial trauma (e.g., wound healing). Inappropriate activation of inflammatory responses is the underlying cause of many common diseases and inflammatory reaction are, therefore, also an important target for drug development.

The most prominent systemic manifestation of inflammation is an elevation of body temperature and a variety of biochemical alterations known as the acute phase reaction which leads to the synthesis of acute phase proteins in the liver.

The local inflammatory reaction is characterized by an initial increase in blood flow to the site of injury, enhanced vascular permeability, and the ordered and directional influx and selective accumulation of different effector cells from the peripheral blood at the site of injury. Influx of antigen non-specific but highly destructive cells (neutrophils) is one of the earliest stages of the inflammatory response. These cells mount a rapid, non-specific phagocytic response.

At a later stage, monocyte-macrophages and cells of other lymphocyte lineages (specific subsets of T-cells and B-cells) appear at the site of injury. These cell types are associated with antigen-specific and more tightly regulated immune responses and once activated also produce protective and inflammatory molecules. An exudation of plasma into the lesion in the early stage is observed also.

Inflammatory cells express increasing numbers of cell-surface proteins and glycoproteins known as cell adhesion molecules. Endothelial cells are also activated during the initial phase of the inflammatory response and then express, among other things, adhesion molecule counter-receptors. The regulated expression of these molecules allows for the precise trafficking of circulating leukocytes to inflammatory sites. Cellular attachment of immune cells to endothelial cells lining blood vessels surrounding the inflammatory site prevents them from being swept past the site of infection or tissue damage and is a crucial step required for the subsequent emigration of these cells into the surrounding inflammatory tissues (extravasation).

The highly efficient process of cellular influx to inflammatory sites is mediated by a plethora of mediator substances supporting and dispersing inflammation. These mediators are found in the serum or tissue fluids, are released by degranulating cells, and are secreted also by inflammatory cells upon activation, or activated endothelial cells in blood vessels at the site of inflammation. They serve as muscle-active and edema-promoting substances, chemotaxins, and cellular activators and inducers of all kinds of effector cells engaged in the inflammatory response.

Inflammatory mediators include some well studied compounds such as anaphylatoxins of the complement cascade, kinins of the coagulation system, leukotrienes, prostaglandins, and many other lipid mediators. Another group of mediators are neuropeptides such as Tachykinins, VIP (vasoactive intestinal peptide), and VPF (vascular permeability factor). These substances enhance capillary permeability and have vasodilatory and bronchoconstrictory activity and also increase the production of mucus.

A number of cytokines, known collectively aspro-inflammatory cytokines because they accelerate inflammation, also regulate inflammatory reactions either directly or by their ability to induce the synthesis of cellular adhesion molecules or other cytokines in certain cell types. The major pro-inflammatory cytokines that are responsible for early responses are IL1-alpha, IL1-beta, IL6, and TNF-alpha. Other pro-inflammatory mediators include LIF, IFN-gamma, OSM, CNTF, TGF-beta, GM-CSF, IL11, IL12, IL17, IL18, IL8 and a variety of other chemokines that chemoattract inflammatory cells, and various neuromodulatory factors. The net effect of an inflammatory response is determined by the balance between pro-inflammatory cytokines and anti-inflammatory cytokines (for example IL4, IL10, and IL13, IL16, IFN-alpha, TGF-beta, IL1ra, G-CSF, soluble receptors for TNF or IL6).

The observed redundancy among the different cytokines and other mediators of inflammation generally guarantees a substitution or complementation of individual components that may have been inactivated under pathological conditions.

Under normal circumstances these cascades of inflammatory reactions induced by the mediators are strictly regulated. Failure to do so can lead to multiple organ failure (e.g., “SIRS” or “systemic inflammatory response syndrome”). Inflammatory mediators and suitable inhibitors are, therefore, of key interest for modulating and ameliorating the effects of inflammatory reactions and their sequelae.

Sepsis/SIRS is an example of an acute systemic inflammatory response to a variety of noxious insults (particularly insults of an infectious origin such as a bacterial infection, but also non-infectious insults are well known and often seen). The systemic inflammatory response seen with sepsis/SIRS is caused by immunological processes that are activated by a variety of immunological mediators such as cytokines, chemokines, nitric oxide, and other immune mediating chemicals of the body. These immunological mediators are generally seen to cause the life-threatening systemic disease seen with sepsis/SIRS. These immunological mediators are, one the one hand, required locally, for example as effective antibacterial response, but are, in contrast, potentially toxic when secreted into the circulation. When secreted into the circulation, these mediators can cause, in an upward spiral of cause and effect, the further systemic release of these mediators, in the end leading to severe disease, such as multiple organ failure and death.

Central in the development of sepsis/SIRS in a subject is the presence and effects of immunological mediators that give rise to a disease that pertains to or affects the body as a whole, a systemic disease. This systemic immunological response can be caused by a variety of clinical insults, such as trauma, burns and pancreatitis. The phrase “systemic inflammatory response syndrome (SIRS)” has been introduced to designate the signs and symptoms of patients suffering from such a condition. SIRS has a continuum of severity ranging from the presence of tachycardia, tachypnea, fever and leukocytosis, to refractory hypotension and, in its most severe from, shock and multiple organ system dysfunction.

The crucial pathophysiologic event that precipitates systemic inflammation is tissue damage. This can occur both as a result of the direct injury to tissues from mechanical or thermal trauma as well as cellular injury induced by mediators of ischemia-reperfusion injury such as oxygen free radicals. Injury results in the acute release of proinflammatory cytokines. If injury is severe, such as in extensive thermal injury, a profound release of cytokines occurs, resulting in the induction of a systemic inflammatory reaction. The ability of the host to adapt to this systemic inflammatory response is dependent on the magnitude of the response, the duration of the response, and the adaptive capacity of the host.

The immune system is a complicated network. Soluble mediators secreted by immune and vascular endothelial cells regulate many immune functions and serve as means of communication between different parts of the system. Mediators are involved in the regulation of their own release as well as in the production and secretion of other mediators. The existence of a network also explains why administration of a specific mediator might trigger systemic inflammation. Therapeutic intervention at different steps might be successful in the prevention of SIRS if the mediator plays a pivotal role in the development of the systemic inflammatory response. However, interventions aimed at neutralizing single mediators of SIRS such as tumor necrosis factor α (TNFα), interleukin-1 (IL-10, and platelet activating factor have in general not been successful in clinical trials. Several proinflammatory cytokines, chemokines, and non-cytokine inflammatory mediators play a role in the pathogenesis of SIRS. Cytokines comprise a broad group of polypeptides with varied functions within the immune response.

The classical mediator of systemic inflammation is TNFα. TNFα is released primarily by macrophages within minutes of local or systemic injury and modulates a variety of immunologic and metabolic events. At sites of local infection or inflammation, TNFα initiates an immune response that activates antimicrobial defense mechanisms and, once the infection is eradicated, tissue repair. It is a potent activator of neutrophils and mononuclear phagocytes, and also serves as a growth factor for fibroblasts and as an angiogenesis factor. However, systemic release of TNFα can precipitate a destructive cascade of events that can result in tissue injury, organ dysfunction and, potentially, death. Among the systemic effects of TNFα are the induction of fever, stimulation of acute phase protein secretion by the liver, activation of the coagulation cascade, myocardial suppression, and induction of systemic vasodilators with resultant hypotension, catabolism, and hypoglycemia. Tumor necrosis factor α is also a potent stimulus for the release of other inflammatory mediators, particularly IL-1 and IL-6. Interleukin-1 is released primarily by mononuclear phagocytes and its physiologic effects are essentially identical to those of TNFα. However, important differences between the functions of IL-1 and TNFα exist. Most notably, IL-1 does not induce tissue injury or apoptotic cell death by itself but can potentiate the injurious effects of TNFα.

Interleukin-6 is another protein that is commonly increased in the circulation of patients with SIRS. This protein is secreted by macrophages, endothelial cells, and fibroblasts. Interleukin-6 itself does not induce tissue injury but its presence in the circulation has been associated with poor outcome in trauma patients, probably because it is a marker of ongoing inflammation. Furthermore, interferon γ (IFN-γ) is a cytokine involved in the amplification of the acute inflammatory response, particularly the stimulation of cytokine secretion, phagocytosis, and respiratory burst activity by macrophages. Interferon γ is secreted primarily by T lymphocytes and natural killer (NK) cells in response to antigen presentation as well as macrophage-derived cytokines such as IL-12 and IL-18. The primary effect of IFN-γ is to amplify the inflammatory response of macrophages. In response to IFN-γ, the phagocytic and respiratory burst activity of macrophages are increased, secretion of inflammatory mediators such as TNF-α and IL-1 are enhanced, and antigen presentation is potentiated by up-regulation of class II major histocompatibility complex.

A central feature in the up-regulation of many of the soluble mediators described above is the transcription factor nuclear factor-κB (NF-κB). NF-κB is comprised of a family of proteins including p50 (NF-κB1), P65 (RelA), C-Rel, and p52 (NF-κB2) that combine to form homo- or heterodimers and ultimately function to regulate the transcription of a variety of cytokine, chemokine, adhesion molecule, and enzyme genes involved in SIRS. The binding of TNFα or IL-1β to their receptors activates a signal transduction cascade a variety of inflammation-associated gene products and modulates their expression. Increased activation of NF-κB has been associated with poor outcome in some studies.

Several non-cytokine factors have been implicated in the pathogenesis of systemic inflammation. Platelet activating factor (PAF) is a phospholipid autocoid release by endothelial cells that regulates the release of cytokines and amplifies the proinflammatory response. Leukotrienes (LTC-LTE) induce contraction of endothelial cells and encourage capillary leakage. Thromboxane A, a macrophage and platelet-derived factor, promotes platelet aggregation, vasoconstriction and, potentially, tissue thrombosis. The complement cascade is comprised of more than 30 proteins that interact in a complex fashion to mediate inflammation and direct lysis of microbes and other cells. Excessive complement activation appears to cause significant cellular injury in the host. Products of the complement cascade, most notably C3a and C5a, are potent activators of inflammation and leukocytes chemotaxis. C3a and C5a also directly activate neutrophils and promote release of reactive oxygen intermediates and proteases. Despite our increased understanding of the role of inflammatory mediators in the pathogenesis of SIRS, most anti-inflammatory drug regimes have had little success in the treatment of this problem. Neutralizing approaches to several inflammatory mediators have been studied. All of these studies have demonstrated, at best, marginal improvement in septic morbidity and mortality.

One of the most widely studied approaches for the treatment of SIRS is the use of monoclonal antibodies to TNFα. Several multicenter, prospective, clinical trials have been undertaken in septic patients using several different antibodies to TNFα. These studies did not demonstrate improved outcome in patients receiving anti-TNFα compared to placebo. One recent study evaluated the efficacy of a chimeric antibody to TNF in patients with severe sepsis.

Circulating levels of TNFα as well as a variety of other inflammatory mediators were assessed. Although circulating levels of TNFα were transiently decreased, anti-TNFα therapy did not result in reduction of circulating levels of other inflammatory mediators such as IL-1, IL-1ra, sTNFR, or IL-6. In addition, evidence of systemic inflammation was not decreased and overall mortality was not improved in anti-TNFα treated patients. Because the relative ineffectiveness of anti-inflammatory therapy aimed at neutralizing single mediators, more broad-based strategies with the goal of neutralizing, removing, or inhibiting the production of several inflammatory mediators are looked forward to. The use of glucocorticoids in the treatment of sepsis has been proposed for more than 30 years. Overall, the use of glucocorticoids to treat sepsis and septic shock has not been beneficial. In many studies, the use of glucocorticoids in septic patients was associated with increased mortality. In burned patients, there is no evidence that administration of glucocorticoids provides effective treatment for systemic inflammation.

Reasons for the lack of efficacy of these agents are likely to be multifactorial. Firstly, the inflammatory response to injury and sepsis is mediated by a complex array of mediators that are largely interrelated. Therefore, blocking or neutralization of a single mediator is not likely to have a marked effect on the overall response. Secondly, the same mediators that are important in inducing tissue injury also play an important role in antimicrobial immunity. Blockade of these mediators may leave the host more susceptible to subsequent infection. Thirdly, many of the mediators, particularly TNFα and IL-1β, are released within minutes of the injury and mobilize the inflammatory cascade shortly thereafter.

Paradoxically, a state of immunosuppression often follows or co-exists with SIRS. The counter anti-inflammatory response syndrome (CARS) appears to be an adaptive mechanism designed to limit the injurious effects of systemic inflammation. However, this response may also render the host more susceptible to systemic infection due to impaired antimicrobial immunity. CARS is often seen after serious trauma. Virtually all components of the immune response have been found to be depressed following injury including macrophage, lymphocyte, and neutrophil function; delayed type hypersensitivity (DTH) responses, immunoglobulin (Ig) and interferon (IFN) production, and serum opsonic capacity. Serum peptides, which suppress lymphocyte proliferation in vitro, have been defined, and the immunosuppressive role of excessive complement activation has also been recognized.

Immune failure occurs early after trauma and the rapidity with which immune function returns to normal may be the best indicator of clinical recovery. Indeed, immediate down-regulation of the immune response may be a protective mechanism for the host, lest too vigorous an early host response creates a catabolic situation incompatible with early survival. The surface expression of the class II HLA-DR on peripheral blood monocytes was measured in 60 patients and was depressed in most, immediately following severe trauma and during subsequent sepsis. However, when patients were grouped according to clinical outcome (uneventful recovery, major infection, and death) an interesting pattern arose. The percentage of monocytes that expressed the HLA-DR antigen returned to the normal range by one week in the first group, by three weeks in those with major infection, but never in those who eventually died. Thus, antigen expression served as a useful marker, or predictor, of clinical outcome in such patients. When monocytes were incubated with bacterial lipopolysaccharide (LPS), those patients who survived had enhanced HLA-DR antigen expression (stimulated towards the normal range), while monocytes from patients who died were relatively resistant to stimulation. Expression of HLA-DR antigen correlates with the ability of these cells to present foreign antigen and thus initiate a specific immune response.

Provided is a method for treating a subject suspected to suffer from a disease comprising an inflammatory condition and/or a counter-inflammatory condition comprising subjecting the subject to a diagnostic process aimed at determining inflammatory disease stage of the subject further comprising providing the subject with a gene-regulatory peptide or functional analogue thereof depending on the outcome of the determination of disease stage.

In one embodiment, the diagnostic process includes determining the level of a pro-inflammatory cytokine, such as pro-inflammatory cytokines that are responsible for early responses are IL1-alpha, IL1-beta, IL6, and TNF-alpha. Other pro-inflammatory mediators include LIF, IFN-gamma, OSM, CNTF, GM-CSF, IL11, IL12, IL17, IL18, IL8 and a variety of other chemokines that chemoattract inflammatory cells, and various neuromodulatory factors. It is preferred that the diagnostic process includes determining the level of a pro-inflammatory cytokine that is selected from the group of tumor necrosis factor-alpha, interferon-gamma, interleukin-1-beta and interleukin-6. Upon determination of pro-inflammatory cytokine levels, and having determined the disease stage as an essentially inflammatory condition, it is herein also provided to treat the subject with a gene-regulatory peptide or functional analogue that down-regulates translocation and/or activity of pro-inflammatory cytokine gene expression mediated by a gene transcription factor. It is for example useful, when the gene transcription factor comprises an NF-kappaB/Rel protein, to inhibit translocation and/or activity of the NF-kappaB/Rel protein. Such inhibition is preferably achieved with a gene-regulatory peptide selected from the group of peptides having NFkappaB down-regulating activity in LPS stimulated RAW264.7 cells.

In another embodiment, the diagnostic process includes determining the level of a counter-inflammatory cytokine which may however be combined with determining for example TNF-alpha levels, to corroborated the diagnostic process. In one embodiment the diagnostic process includes determining the level of a counter-inflammatory cytokine, such as for example IL4, IL10, and IL13, IL16, IFN-alpha, IL1ra, G-CSF, soluble receptors for TNF or IL6. It is preferred that the counter-inflammatory cytokine is selected from the group of interleukin-4 and interleukin-10.

An essentially inflammatory condition is preferably characterized by elevated levels of at least one, but preferably at least two or three pro-inflammatory cytokines for example produced by circulating polymorph bone marrow cells (PBMCs), such as elevated plasma or serum levels of one or more of the pro-inflammatory cytokines that are responsible for early responses such as IL1-alpha, IL1-beta, IL6, and TNF-alpha. Other pro-inflammatory mediators include LIF, IFN-gamma, OSM, CNTF, GM-CSF, IL11, IL12, IL17, IL18, IL8. As each diagnostician may have his or her own preference for testing an inflammatory mediator, as said, it is preferred that the diagnostic process is based on determining the level of a pro-inflammatory cytokine that is selected from the group of tumor necrosis factor-alpha, interferon-gamma, interleukin-1-beta and interleukin-6. PBMCs of patients in an essentially inflammatory condition in general produce plasma levels of at least >2 SD above the control mean (CM) values of TNF-alpha, IL-1beta, and/or IL-6 produced by control PBMCs from non-diseased individuals of comparable age and background. However, testing other pro-inflammatory mediators in other test samples may be preferred by the individual diagnostician based on his or her own experience and preferences.

An essentially counter-inflammatory condition is preferably characterized by elevated levels of at least one, but preferably at least two or three counter-inflammatory cytokines produced by circulating PBMCs, such as elevated plasma levels of one or more of the a counter-inflammatory cytokine, such as for example IL4, IL10, and IL13, IL6, IFN-alpha, IL1ra, G-CSF, soluble receptors for TNF or IL6. It is preferred that the counter-inflammatory cytokine is selected from the group of interleukin-4 and interleukin-10. PBMCs of patients in an essentially counter-inflammatory condition in general produce at least >2 SD above the control mean (CM) values of IL-4 or IL-10 produced by control PBMCs from non-diseased individuals of comparable age and background. However, testing other counter-inflammatory mediators in other test samples may be preferred by the individual diagnostician based on his or her own experience and preferences.

To validate a diagnosis of an essentially inflammatory condition, it may be useful in the diagnostic process to also determine the levels of one or more counter-inflammatory mediators, and vice-versa. Useful are diagnostic tests such as flow cytometry assays of serum/plasma/supernatant available with BD Biosciences as a Cytometric Bead Array (CBA), for example a CBA human Th1/Th2 cytokine kit for the measurement of IL-2, IL-4, IL-5, IL-10, TNF-a and IFN-g in a single sample or available with Biosource International, the Biosource Multiplex antibody Bead Kit for measurement of IL-1b, IL-2, IL-4, IL-5, IL-6, IL-8, IL-10, IFN-g, TNF-a and GM-CSF in single sample or for example the Biosource cytoset ELISA for measurement of individual cytokines and soluble DR4. Inflammation mediators could be also measured by HPLC with the help of FITC labeled specific antibodies.

It is herein also provided to use a diagnostic process that includes determining HLA-DR expression on circulating monocytes of the subject. HLA-DR antigen under expression indicates a counter-inflammatory condition and is a discriminator of poor clinical outcome. Such a patient is then treated with a gene-regulatory peptide that up-regulates pro-inflammatory cytokine gene expression. Such a peptide is preferably selected from the group of peptides having NFkappaB up-regulating activity in LPS stimulated RAW264.7 cells.

In another embodiment, provided is using a diagnostic process that includes determining arachidonic acid metabolite levels in the subject or a diagnostic process that includes plasma prostaglandin levels in the subject. In particular, determining a ratio between prostaglandins 1 and 2 (PGE1 and PGE2) is useful, whereby a high PGE2 levels are indicative for an inflammatory condition which should be treated accordingly.

In short, provided is a method of treatment of a subject suspected to suffer from a disease comprising an inflammatory condition and/or a counter-inflammatory condition whereby it is provided to treat the subject with a gene-regulatory peptide or functional analogue thereof capable of down-regulation of inflammation after having determined disease stage as an essentially inflammatory condition. A subject with an essentially inflammatory condition is preferably treated with a gene-regulatory peptide that down-regulates translocation and/or activity of pro-inflammatory cytokine gene expression mediated by a gene transcription factor. It is preferred that the gene transcription factor comprises an NF-kappaB/Rel protein whereby translocation and/or activity of the NF-kappaB/Rel protein is inhibited by the peptide. Such a peptide is preferably selected from the group of peptides having NFkappaB down-regulating activity in LPS stimulated RAW264.7 cells, and even more preferably from the group of peptides having NFkappaB down-regulating activity in LPS unstimulated RAW264.7 cells.

Also, provided is a method of treatment of a subject suspected to suffer from a disease comprising an inflammatory condition and/or a counter-inflammatory condition whereby it is provided to treat the subject with a gene-regulatory peptide or functional analogue thereof capable of up-regulation of inflammation after having determined disease stage as an essentially counter-inflammatory condition. A subject with an essentially counter inflammatory condition is preferably treated with a gene-regulatory peptide that up-regulates translocation and/or activity of pro-inflammatory cytokine gene expression mediated by a gene transcription factor. In one embodiment, it is preferred to treat the counter-inflammatory condition with a gene-regulatory peptide that activates an NF-kappaB/Rel protein. Than it is preferred that translocation and/or activity of the NF-kappaB/Rel protein is activated. Such a peptide is preferably selected from the group of peptides having NFkappaB up-regulating activity in LPS un-stimulated RAW264.7 cells, more preferably from the group of peptides having NFkappaB up-regulating activity in LPS stimulated RAW264.7 cells.

Also provided is the use of a gene-regulatory peptide or functional analogue thereof for the production of a pharmaceutical composition for the treatment of a disease comprising an inflammatory condition and/or a counter-inflammatory condition wherein a subject suffering from the disease is subjected to a diagnostic process aimed at determining inflammatory disease stage of the subject and where treatment is selected depending on the outcome of the determination of disease stage, for example wherein the diagnostic process includes determining the level of a pro-inflammatory cytokine and the pro-inflammatory cytokine is selected from the group of tumor necrosis factor-alpha, interferon-gamma, interleukin-1-beta and interleukin-6.

When the diagnostic process includes determining the level of a counter-inflammatory cytokine it is preferred that counter-inflammatory cytokine is selected from the group of interleukin-4 and interleukin-10.

Provided is treating the subject with a gene-regulatory peptide or functional analogue thereof based on the determination of disease stage as an essentially inflammatory condition. It is than preferred to treat the subject with a gene-regulatory peptide down-regulates translocation and/or activity of pro-inflammatory cytokine gene expression mediated by a gene transcription factor, preferably selected from the group of peptides or analogues having NFkappaB down-regulating activity in LPS stimulated RAW264.7 cells.

On the other hand, when treatment is selected on the basis of the determination of disease stage as an essentially counter-inflammatory condition, the treatment preferably is done with a gene-regulatory peptide that up-regulates translocation and/or activity of pro-inflammatory cytokine gene expression mediated by a gene transcription factor, such as with a peptide is selected from the group of peptides having NFkappaB up-regulating activity in LPS un-stimulated RAW264.7 cells.

The treatment of sepsis/SIRS, as is for example provided herein, is in one embodiment directed at inhibiting the production and release of immune mediators involved in the generation of sepsis/SIRS, thereby blocking the upward spiral of SIRS. Inhibiting the production of these mediators is achieved by regulating particular gene transcription activators with a gene-regulatory peptide as provided herein. A particular family of gene transcription activators, generally and widely known to be central in the activation of genes leading to the production of immunological mediators, is the NF-kappaB protein family. The ability to inhibit the NF-kappaB protein family is currently a widely sought after desideratum for the development of immunomodulating therapeutic approaches, the family being so central in shaping a wide array of immune responses of the body. Gene-regulatory peptides as herein have the ability to inhibit proteins of this family.

Provided is a method for treating a subject suspected to suffer from a disease comprising an inflammatory condition and/or a counter-inflammatory condition comprising subjecting the subject to a diagnostic process aimed at determining inflammatory disease stage of the subject further comprising providing the subject with a gene-regulatory peptide or functional analogue thereof depending on the outcome of the determination of disease stage wherein the subject is provided with a peptide selected from the group of LQGV (SEQ ID NO:1), MTR, MTRV (SEQ ID NO:20), AQGV (SEQ ID NO:2), LAGV (SEQ ID NO:10), AQG, LQG, VLPALPQ (SEQ ID NO:13), LAG, and VLPALP (SEQ ID NO:4), preferably wherein the peptide is selected from the group of LQGV (SEQ ID NO:1), MTR, MTRV (SEQ ID NO:20), AQGV (SEQ ID NO:2), LAGV (SEQ ID NO:10), AQG, and LQG, most preferably wherein the peptide is selected from the group of LQGV (SEQ ID NO:1), LAGV (SEQ ID NO:10) and AQGV (SEQ ID NO:2) or wherein the peptide is selected from the group of LQGV (SEQ ID NO:1), MTR and MTRV (SEQ ID NO:20).

### EXAMPLES

In a particular embodiment, the invention relates to treatment of the systemic inflammatory response seen with sepsis/SIRS/CARS and caused by the immune mediators, the treatment comprising inhibiting the production and effects of the mediators by inhibiting gene expression, in particular by inhibiting gene expression regulated via the NF-kappaB protein family. It is also realized that defining patients at very high risk of infection and multi-system organ failure before either develop, is paramount to the introduction and interpretation of clinical treatment in this area.

A gene regulatory peptide is preferably a short peptide, preferably of at most 30 amino acids long, or a functional analogue or derivative thereof. In a much preferred embodiment, the peptide is from about three to about 15 amino acids long, preferably four to twelve, more preferably four to nine, most preferably four to six amino acids long, or a functional analogue or derivative thereof. Of course, such a gene-regulatory peptide can be longer, for example by extending it (N- and/or C-terminally), with more amino acids or other side groups, which can for example be (enzymatically) cleaved off when the molecule enters the place of final destination. In particular a method is provided wherein the gene-regulatory peptide modulates translocation and/or activity of a gene transcription factor. It is particularly useful when the gene transcription factor comprises an NF-kappaB/Rel protein or an AP-1 protein. Insults generally induce increased expression of inflammatory cytokines due to activation of NF-κB and AP-1, and in a preferred embodiment provided is a method wherein translocation and/or activity of the NF-kappaB/Rel protein is inhibited. In one embodiment, the peptide is selected from the group of peptides LQG, AQG, LQGV (SEQ ID NO:1), AQGV (SEQ ID NO:2), LQGA, VLPALP (SEQ ID NO:4), ALPALP (SEQ ID NO:5), VAPALP (SEQ ID NO:6), ALPALPQ (SEQ ID NO:7), VLPAAPQ (SEQ ID NO:8), VLPALAQ (SEQ ID NO:9), LAGV (SEQ ID NO:10), VLAALP (SEQ ID NO:11), VLPALA (SEQ ID NO:12), VLPALPQ (SEQ ID NO:13), VLAALPQ (SEQ ID NO:14), VLPALPA (SEQ ID NO:15), GVLPALPQ (SEQ ID NO:16), LQGVLPALPQVVC (SEQ ID NO:17), LPGCPRGVNPVVS (SEQ ID NO:18), LPGC (SEQ ID NO:19), MTRV (SEQ ID NO:20), MTR, VVC. Insults often induce increased expression of inflammatory cytokines due to activation of NF-κB and AP-1.

Inflammatory cytokines can be expressed by epithelium, perivascular cells and adherent or transmigrating leukocytes, inducing numerous pro-inflammatory and procoagulant effects. Together these effects predispose to inflammation, thrombosis and hemorrhage. Of clinical and medical interest and value, the present invention provides the opportunity to selectively control NFκB-dependent gene expression in tissues and organs in a living subject, preferably in a primate, allowing up-regulating essentially anti-inflammatory responses such as IL-10, and down-regulating essentially pro-inflammatory responses such as mediated by TNF-alpha, nitric oxide (NO), IL-5, IL-1beta.

Thus provided is use of a NFκB regulating peptide or derivative thereof for the production of a pharmaceutical composition for the treatment of an inflammatory condition, preferably in a primate, and provides a method of treatment of an inflammatory condition, notably in a primate. It is preferred when the treatment directed against an inflammatory condition comprises administering to the subject a pharmaceutical composition comprising an NFkappaB down-regulating peptide or functional analogue thereof. Examples of useful NFkappaB down-regulating peptides are VLPALPQVVC (SEQ ID NO:21), LQGVLPALPQ (SEQ ID NO:22), LQG, LQGV (SEQ ID NO:1), GVLPALPQ (SEQ ID NO:23), VLPALP (SEQ ID NO:4), VVC, MTR and circular LQGVLPALPQVVC (SEQ ID NO:17). More down-regulating peptides and functional analogues can be found using the methods as provided herein. Most prominent among NFkappaB down-regulating peptides are VLPALPQVVC (SEQ ID NO:21), LQGVLPALPQ (SEQ ID NO:22), LQG, LQGV (SEQ ID NO:1), and VLPALP (SEQ ID NO:4). These are also capable of reducing production of NO by a cell. It is herein also provided to use a composition that comprises at least two oligopeptides or functional analogues thereof, each capable of down-regulation NFkappaB, and thereby reducing production of NO and/or TNF-alpha by a cell, in particular wherein the at least two oligopeptides are selected from the group LQGV (SEQ ID NO:1), AQGV (SEQ ID NO:2) and VLPALP (SEQ ID NO:4), for the treatment of inflammatory condition, and, moreover to treat the systemic inflammatory response often seen in severe burn patients.

Thus provided is use of a NFκB regulating peptide or derivative thereof for the production of a pharmaceutical composition for the treatment of a subject suffering from a counter-inflammatory condition, in particular of a human, and provides a method of treatment of a counter-inflammatory condition. It is preferred when the treatment comprises administering to the subject a pharmaceutical composition comprising an NFkappaB up-regulating peptide or functional analogue thereof. The invention for this purpose provides use of a such signaling molecule comprising a NF-kappaB up-regulating peptide or functional analogue thereof for the production of a pharmaceutical composition for the treatment of a counter anti-inflammatory condition, for example occurring after a traumatic injury of a subject, in particular wherein translocation and/or activity of the NF-kappaB/Rel protein is up-regulated, resulting in stimulating a cascade of cytokine reactions.

In one embodiment, the invention is providing a method and means to treat the systemic immunosuppressive reaction by providing a subject believed to be in need thereof with a pharmaceutical composition comprising a NF-kappaB up-regulating peptide or functional analogue thereof, preferably an NFkappaB regulating peptide such as VLPALPQ (SEQ ID NO:13), GVLPALPQ (SEQ ID NO:16) or MTRV (SEQ ID NO:20), or mixtures of two or three of these peptides.

A gene-regulatory peptide such as an NFkappaB regulating peptide can be given by infusion, the peptide (or analogue) concentration preferably being from about 1 to about 1000 mg/l, but the peptide can also been given in a bolus injection. Doses of 1 to 5 mg/kg bodyweight, for example every eight hours in a bolus injection or by infusion until the patient stabilizes, are recommended. It is preferred to monitor cytokine profiles, such as TNF-alpha or IL-10 levels, in the plasma of the treated patient, and to stop treatment when these levels are considered within normal boundaries.

More gene-regulating peptides and functional analogues can be found in a (bio)assay, such as a NFkappaB translocation assay as provided herein, and a by testing peptides for NFkappaB down- or up-regulating activity in LPS-stimulated or unstimulated RAW264.7 cells. For anti-inflammatory treatment, it is preferred that the peptide is selected from the group of peptides having NFkappaB down-regulating activity in LPS stimulated RAW264.7 cells, especially when the subject is at risk to experience SIRS. For treatment of an immunosuppressed state, such as seen in CARS, it is preferred that the peptide is selected from the group of peptides having NFkappaB up-regulating activity in LPS stimulated RAW264.7 cells, especially when the subject is at risk to experience CARS.

In response to a variety of pathophysiological and developmental signals, the NFκB/Rel family of transcription factors are activated and form different types of hetero- and homodimers among themselves to regulate the expression of target genes containing kappaB-specific binding sites. NF-κB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFκB is a heterodimer of p65 (RELA) and p50 (NF-κB1). Among the activated NFκB dimers, p50-p65 heterodimers are known to be involved in enhancing the transcription of target genes and p50-p50 homodimers in transcriptional repression. However, p65-p65 homodimers are known for both transcriptional activation and repressive activity against target genes. KappaB DNA binding sites with varied affinities to different NFκB dimers have been discovered in the promoters of several eukaryotic genes and the balance between activated NFκB homo- and heterodimers ultimately determines the nature and level of gene expression within the cell.

The term “NFκB-regulating peptide” as used herein refers to a peptide or a modification or derivative thereof capable of modulating the activation of members of the NFκB/Rel family of transcription factors. Activation of NFkB can lead to enhanced transcription of target genes. Also, it can lead to transcriptional repression of target genes. NFκB activation can be regulated at multiple levels. For example, the dynamic shuttling of the inactive NFκB dimers between the cytoplasm and nucleus by IkappaB proteins and its termination by phosphorylation and proteasomal degradation, direct phosphorylation, acetylation of NFκB factors, and dynamic reorganization of NFκB subunits among the activated NFκB dimers have all been identified as key regulatory steps in NFκB activation and, consequently, in NFκB-mediated transcription processes.

Thus, an NFκB-regulating peptide is capable of modulating the transcription of genes that are under the control of NFκB/Rel family of transcription factors. Modulating comprises the up-regulation or the down-regulation of transcription. In a preferred embodiment, a peptide according to the invention, or a functional derivative or analogue thereof is used for the production of a pharmaceutical composition. Such peptides are preferably selected from group of peptides having NFkappaB down-regulating activity in LPS stimulated RAW264.7 cells. Examples of useful NFkappaB down-regulating peptides to be included in such a pharmaceutical composition are VLPALPQVVC (SEQ ID NO:21), LQGVLPALPQ (SEQ ID NO:22), LQG, LQGV (SEQ ID NO:1), GVLPALPQ (SEQ ID NO:23), VLPALP (SEQ ID NO:4), VVC, MTR and circular LQGVLPALPQVVC (SEQ ID NO:17).

More gene-regulating peptides and functional analogues can be found in a (bio)assay, such as a NFkappaB translocation assay as provided herein, which can also be used to further identify peptides having NFkappaB up-regulating activity in LPS stimulated RAW264.7 cells. Most prominent among NFkappaB down-regulating peptides are VLPALPQVVC (SEQ ID NO:21), LQGVLPALPQ (SEQ ID NO:22), LQG, LQGV (SEQ ID NO:1), and VLPALP. These are also capable of reducing production of NO by a cell.

It is herein also provided to use a composition that comprises at least two oligopeptides or functional analogues thereof, each capable of down-regulation NFkappaB, and thereby reducing production of NO and/or TNF-alpha by a cell, in particular wherein the at least two oligopeptides are selected from the group LQGV (SEQ ID NO:1), AQGV (SEQ ID NO:2) and VLPALP (SEQ ID NO:4). Useful NFkappaB up-regulating peptides are VLPALPQ (SEQ ID NO:13), GVLPALPQ (SEQ ID NO:16) and MTRV (SEQ ID NO:20). As indicated, more gene-regulatory peptides may be found with an appropriate (bio)assay. A gene-regulatory peptide as used herein is preferably short. Preferably, such a peptide is three to 15 amino acids long, more preferably, wherein the lead peptide is three to nine amino acids long, most preferred wherein the lead peptide is four to six amino acids long, and capable of modulating the expression of a gene, such as a cytokine, in a cell. In a preferred embodiment, a peptide is a gene-regulatory peptide that is capable of traversing the plasma membrane of a cell or, in other words, a peptide that is membrane-permeable.

Functional derivative or analogue herein relates to the signaling molecular effect or activity as for example can be measured by measuring nuclear translocation of a relevant transcription factor, such as NF-kappaB in an NF-kappaB assay, or AP-1 in an AP-1 assay, or by another method as provided herein. Fragments can be somewhat (i.e., one or two amino acids) smaller or larger on one or both sides, while still providing functional activity. Such a bioassay comprises an assay for obtaining information about the capacity or tendency of a peptide, or a modification thereof, to regulate expression of a gene. A scan with for example a 15-mer, or a 12-mer, or a 9-mer, or a 8-mer, or a 7-mer, or a 6-mer, or a 5-mer, or a 4-mer or a 3-mer peptides can yield valuable information on the linear stretch of amino acids that form an interaction site and allows identification of gene-regulatory peptides that have the capacity or tendency to regulate gene expression. Gene-regulatory peptides can be modified to modulate their capacity or tendency to regulate gene expression, which can be easily assayed in an in vitro bioassay such as a reporter assay. For example, some amino acid at some position can be replaced with another amino acid of similar or different properties. Alanine (Ala)-replacement scanning, involving a systematic replacement of each amino acid by an Ala residue, is a suitable approach to modify the amino acid composition of a gene-regulatory peptide when in a search for a gene-regulatory peptide capable of modulating gene expression. Of course, such replacement scanning or mapping can be undertaken with amino acids other than Ala as well, for example with D-amino acids. In one embodiment, a peptide derived from a naturally occurring polypeptide is identified as being capable of modulating gene expression of a gene in a cell. Subsequently, various synthetic Ala-mutants of this gene-regulatory peptide are produced. These Ala-mutants are screened for their enhanced or improved capacity to regulate expression of a gene compared to gene-regulatory polypeptide.

Furthermore, a gene-regulatory peptide, or a modification or analogue thereof, can be chemically synthesized using D- and/or L-stereoisomers. For example, a gene-regulatory peptide that is a retro-inverso of an oligopeptide of natural origin is produced. The concept of polypeptide retro-inversion (assemblage of a natural L-amino acid-containing parent sequence in reverse order using D-amino acids) has been applied successfully to synthetic peptides. Retro-inverso modification of peptide bonds has evolved into a widely used peptidomimetic approach for the design of novel bioactive molecules which has been applied to many families of biologically active peptide. The sequence, amino acid composition and length of a peptide will influence whether correct assembly and purification are feasible. These factors also determine the solubility of the final product. The purity of a crude peptide typically decreases as the length increases. The yield of peptide for sequences less than 15 residues is usually satisfactory, and such peptides can typically be made without difficulty.

The overall amino acid composition of a peptide is an important design variable. A peptide's solubility is strongly influenced by composition. Peptides with a high content of hydrophobic residues, such as Leu, Val, Ile, Met, Phe and Trp, will either have limited solubility in aqueous solution or be completely insoluble. Under these conditions, it can be difficult to use the peptide in experiments, and it may be difficult to purify the peptide if necessary. To achieve a good solubility, it is advisable to keep the hydrophobic amino acid content below 50% and to make sure that there is at least one charged residue for every five amino acids. At physiological pH Asp, Glu, Lys, and Arg all have charged side chains. A single conservative replacement, such as replacing Ala with Gly, or adding a set of polar residues to the N- or C-terminus, may also improve solubility. Peptides containing multiple Cys, Met, or Trp residues can also be difficult to obtain in high purity partly because these residues are susceptible to oxidation and/or side reactions. If possible, one should choose sequences to minimize these residues. Alternatively, conservative replacements can be made for some residues. For instance, Norleucine can be used as a replacement for Met, and Ser is sometimes used as a less reactive replacement for Cys. If a number of sequential or overlapping peptides from a protein sequence are to be made, making a change in the starting point of each peptide may create a better balance between hydrophilic and hydrophobic residues. A change in the number of Cys, Met, and Trp residues contained in individual peptides may produce a similar effect.

In another embodiment, a gene-regulatory peptide capable of modulating gene expression is a chemically modified peptide. A peptide modification includes phosphorylation (e.g., on a Tyr, Ser or Thr residue), N-terminal acetylation, C-terminal amidation, C-terminal hydrazide, C-terminal methyl ester, fatty acid attachment, sulfonation (tyrosine), N-terminal dansylation, N-terminal succinylation, tripalmitoyl-S-Glyceryl Cysteine (PAM3 Cys-OH) as well as farnesylation of a Cys residue. Systematic chemical modification of a gene-regulatory peptide can for example be performed in the process of gene-regulatory peptide optimization.

Synthetic peptides can be obtained using various procedures known in the art. These include solid phase peptide synthesis (SPPS) and solution phase organic synthesis (SPOS) technologies. SPPS is a quick and easy approach to synthesize peptides and small proteins. The C-terminal amino acid is typically attached to a cross-linked polystyrene resin via an acid labile bond with a linker molecule. This resin is insoluble in the solvents used for synthesis, making it relatively simple and fast to wash away excess reagents and by-products.

The peptides as mentioned in this document such as LQG, AQG, LQGV (SEQ ID NO:1), AQGV (SEQ ID NO:2), LQGA, VLPALP (SEQ ID NO:4), ALPALP (SEQ ID NO:5), VAPALP (SEQ ID NO:6), ALPALPQ (SEQ ID NO:7), VLPAAPQ (SEQ ID NO:8), VLPALAQ (SEQ ID NO:9), LAGV (SEQ ID NO:10), VLAALP (SEQ ID NO:11), VLPALA (SEQ ID NO:12), VLPALPQ (SEQ ID NO:13), VLAALPQ (SEQ ID NO:14), VLPALPA (SEQ ID NO:15), GVLPALPQ (SEQ ID NO:16), VVCNYRDVRFESIRLPGCPRGVNPVVSYAVALSCQCAL (SEQ ID NO:24), RPRCRPINATLAVEKEGCPVCITVNTTICAGYCPT (SEQ ID NO:25), SKAPPPSLPSPSRLPGPS (SEQ ID NO:26), LQGVLPALPQVVC (SEQ ID NO:17), SIRLPGCPRGVNPVVS (SEQ ID NO:27), LPGCPRGVNPVVS (SEQ ID NO:18), LPGC (SEQ ID NO:19), MTRV (SEQ ID NO:20), MTR, and VVC may be prepared by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the alpha-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DWEA) in DMF/NMP.

Upon completion of the synthesis, the peptide resin was treated with a mixture of trifluoroacetic acid (TFA)/H₂O/triisopropylsilane (TIS) 95:2.5:2.5. After 30 minutes TIS was added until decolorization. The solution was evaporated in vacuo and the peptide precipitated with diethyl ether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/minute; absorbance was detected from 190-370 nm. There were different gradient systems used. For example for peptides LQG and LQGV: ten minutes 100% A followed by linear gradient 0-10% B in 50 minutes. For example for peptides VLPALP (SEQ ID NO:4) and VLPALPQ: five minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

RAW 264.7 macrophages, obtained from American Type Culture Collection (Manassas, Va.), were cultured at 37° C. in 5% CO₂ using DMEM containing 10% FBS and antibiotics (100 U/ml of penicillin, and 100 μg/ml streptomycin). Cells (1×10⁶/ml) were incubated with peptide (10 μg/ml) in a volume of 2 ml. After eight hours of cultures cells were washed and prepared for nuclear extracts.

Nuclear extracts and EMSA were prepared according to Schreiber et al. Methods (Schreiber et al. 1989, Nucleic Acids Research 17). Briefly, nuclear extracts from peptide stimulated or nonstimulated macrophages were prepared by cell lysis followed by nuclear lysis. Cells were then suspended in 400 μl of buffer (10 mM HEPES (pH 7.9), 10 mM KCl, 0.1 mM KCL, 0.1 mM EDTA, 0.1 mM EGTA, 1 mM DTT, 0.5 mM PMSF and protease inhibitors), vigorously vortexed for 15 seconds, left standing at 4° C. for 15 minutes, and centrifuged at 15,000 rpm for two minutes. The pelleted nuclei were resuspended in buffer (20 mM HEPES (pH 7.9), 10% glycerol, 400 mM NaCl, 1 mM EDTA, 1 mM EGTA, 1 mM DTT, 0.5 mM PMSF and protease inhibitors) for 30 minutes on ice, then the lysates were centrifuged at 15,000 rpm for two minutes. The supernatants containing the solubilized nuclear proteins were stored at −70° C. until used for the Electrophoretic Mobility Shift Assays (EMSA).

Electrophoretic mobility shift assays were performed by incubating nuclear extracts prepared from control (RAW 264.7) and peptide treated RAW 264.7 cells with a ³²P-labeled double-stranded probe (5′ AGCTCAGAGGGGGACTTTCCGAGAG 3′ (SEQ ID NO:28)) synthesized to represent the NF-kappaB binding sequence. Shortly, the probe was end-labeled with T4 polynucleotide kinase according to manufacturer's instructions (Promega, Madison, Wis.). The annealed probe was incubated with nuclear extract as follows: in EMSA, binding reaction mixtures (20 μl) contained 0.25 μg of poly(dI-dC) (Amersham Pharmacia Biotech) and 20,000 rpm of ³²P-labeled DNA probe in binding buffer consisting of 5 mM EDTA, 20% Ficoll, 5 mM DTT, 300 mM KCl and 50 mM HEPES. The binding reaction was started by the addition of cell extracts (10 μg) and was continued for 30 minutes at room temperature. The DNA-protein complex was resolved from free oligonucleotide by electrophoresis in a 6% polyacrylamide gel. The gels were dried and exposed to x-ray films.

The transcription factor NF-κB participates in the transcriptional regulation of a variety of genes. Nuclear protein extracts were prepared from LPS and peptide treated RAW264.7 cells or from LPS treated RAW264.7 cells. In order to determine whether the peptide modulates the translocation of NF-κB into the nucleus, on these extracts EMSA was performed. Here we see that indeed some peptides are able to modulate the translocation of NF-κB since the amount of labeled oligonucleotide for NF-κB is reduced. In this experiment peptides that show the modulation of translocation of NF-κB are: VLPALPQVVC (SEQ ID NO:21), LQGVLPALPQ (SEQ ID NO:22), LQG, LQGV (SEQ ID NO:1), GVLPALPQ (SEQ ID NO:23), VLPALP (SEQ ID NO:4), VLPALPQ (SEQ ID NO:13), GVLPALPQ (SEQ ID NO: 16), VVC, MTRV (SEQ ID NO:20), MTR.

RAW 264.7 mouse macrophages were cultured in DMEM, containing 10% or 2% FBS, penicillin, streptomycin and glutamine, at 37° C., 5% CO₂. Cells were seeded in a 12-well plate (3×10⁶ cells/ml) in a total volume of 1 ml for two hours and then stimulated with LPS (E. coli 026:B6; Difco Laboratories, Detroit, Mich., USA) and/or peptide (1 microgr/ml). After 30 minutes of incubation plates were centrifuged and cells were collected for nuclear extracts. Nuclear extracts and EMSA were prepared according to Schreiber et al. Cells were collected in a tube and centrifuged for five minutes at 2000 rpm (rounds per minute) at 4° C. (Universal 30 RF, Hettich Zentrifuges). The pellet was washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five microliters 10% NP-40 was added and the sample was centrifuged (two minutes, 4000 rpm, 4° C.). The supernatant (cytoplasmic fraction) was collected and stored at −70° C. The pellet, which contains the nuclei, was washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples were left to shake at 4° C. for at least 60 minutes. Finally the samples were centrifuged and the supernatant (nucleic fraction) was stored at −70° C.

Bradford reagent (Sigma) was used to determine the final protein concentration in the extracts. For Electrophoretic mobility shift assays an oligonucleotide representing NF-κB binding sequence (5′-AGC TCA GAG GGG GAC TTT CCG AGA G-3′ (SEQ ID NO:28)) was synthesized. Hundred pico mol sense and antisense oligo were annealed and labeled with γ-³²P-dATP using T4 polynucleotide kinase according to the manufacturer's instructions (Promega, Madison, Wis.). Nuclear extract (5-7.5 μg) was incubated for 30 minutes with 75000 cpm probe in binding reaction mixture (20 microliters) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. The DNA-protein complex was resolved from free oligonucleotide by electrophoresis in a 4-6% polyacrylamide gel (150 V, two to four hours). The gel was then dried and exposed to x-ray film. The transcription factor NF-κB participates in the transcriptional regulation of a variety of genes. Nuclear protein extracts were prepared from either LPS (1 mg/ml), peptide (1 mg/ml) or LPS in combination with peptide treated and untreated RAW264.7 cells. In order to determine whether the peptides modulate the translocation of NF-κB into the nucleus, on these extracts EMSA was performed. Peptides are able to modulate the basal as well as LPS induced levels of NF-κB. In this experiment peptides that show the inhibition of LPS induced translocation of NF-κB are: VLPALPQVVC (SEQ ID NO:21), LQGVLPALPQ (SEQ ID NO:22), LQG, LQGV (SEQ ID NO:1), GVLPALPQ (SEQ ID NO:23), VLPALP (SEQ ID NO:4), VVC, MTR and circular LQGVLPALPQVVC (SEQ ID NO:17). Peptides that in this experiment promote LPS induced translocation of NF-κB are: VLPALPQ (SEQ ID NO:13), GVLPALPQ (SEQ ID NO:16) and MTRV (SEQ ID NO:20). Basal levels of NF-κB in the nucleus was decreased by VLPALPQVVC (SEQ ID NO:21), LQGVLPALPQ (SEQ ID NO:22), LQG and LQGV (SEQ ID NO:1) while basal levels of NF-κB in the nucleus was increased by GVLPALPQ (SEQ ID NO:23), VLPALPQ (SEQ ID NO:13), GVLPALPQ (SEQ ID NO:16), VVC, MTRV (SEQ ID NO:20), MTR and LQGVLPALPQVVC (SEQ ID NO:17). In other experiments, QVVC (SEQ ID NO:29) also showed the modulation of translocation of NF-κB into nucleus (data not shown).

### Further Modes of Identification of Gene-Regulatory Peptides by NFκB Analysis

Cells: Cells will be cultured in appropriate culture medium at 37° C., 5% CO₂. Cells will be seeded in a 12-well plate (usually 1×10⁶ cells/ml) in a total volume of 1 ml for two hours and then stimulated with regulatory peptide in the presence or absence of additional stimuli such as LPS. After 30 minutes of incubation plates will be centrifuged and cells are collected for cytosolic or nuclear extracts.

Nuclear Extracts: Nuclear extracts and EMSA could be prepared according to Schreiber et al. Method (Schriber et al. 1989, Nucleic Acids Research 17). Cells are collected in a tube and centrifuged for five minutes at 2000 rpm (rounds per minute) at 4° C. (Universal 30 RF, Hettich Zentrifuges). The pellet is washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five microliters 10% NP-40 is added and the sample is centrifuged (two minutes, 4000 rpm, 4° C.). The supernatant (cytoplasmic fraction) was collected and stored at −70° C. for analysis. The pellet, which contains the nuclei, is washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples are left to shake at 4° C. for at least 60 minutes. Finally the samples are centrifuged and the supernatant (nucleic fraction) is stored at −70° C. for analysis.

Bradford reagent (Sigma) could be used to determine the final protein concentration in the extracts.

EMSA: For Electrophoretic mobility shift assays an oligonucleotide representing NF-κB binding sequence such as (5′-AGC TCA GAG GGG GAC TTT CCG AGA G-3′ (SEQ ID NO:28)) are synthesized. Hundred pico mol sense and antisense oligo are annealed and labeled with γ-³²P-dATP using T4 polynucleotide kinase according to the manufacturer's instructions (Promega, Madison, Wis.). Cytosolic extract or nuclear extract (5-7.5 μg) from cells treated with regulatory peptide or from untreated cells is incubated for 30 minutes with 75000 cpm probe in binding reaction mixture (20 microl) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. Or cytosolic and nuclear extract from untreated cells or from cells treated with stimuli could also be incubated with probe in binding reaction mixture and binding buffer. The DNA-protein complex is resolved from free oligonucleotide by electrophoresis in a 4-6% polyacrylamide gel (150 V, two to four hours). The gel is then dried and exposed to x-ray film. Peptides can be biotinylated and incubated with cells. Cells are then washed with phosphate-buffered saline, harvested in the absence or presence of certain stimulus (LPS, PHA, TPA, anti-CD3, VEGF, TSST-1, VIP or know drugs etc.). After culturing cells are lysed and cells lysates (whole lysate, cytosolic fraction or nuclear fraction) containing 200 micro gram of protein are incubated with 50 microliters Neutr-Avidin-plus beads for one hour at 4° C. with constant shaking. Beads are washed five times with lysis buffer by centrifugation at 6000 rpm for one minute. Proteins are eluted by incubating the beads in 0.05 N NaOH for one minute at room temperature to hydrolyze the protein-peptide linkage and analyzed by SDS-polyacrylamide gel electrophoresis followed by immunoprecipitated with agarose-conjugated anti-NF-κB subunits antibody or immunoprecipitated with antibody against to be studied target. After hydrolyzing the protein-peptide linkage, the sample could be analyzed on HPLS and mass-spectrometry. Purified NF-κB subunits or cell lysate interaction with biotinylated regulatory peptide can be analyzed on biosensor technology. Peptides can be labeled with FITC and incubated with cells in the absence or presence of different stimulus. After culturing, cells can be analyzed with fluorescent microscopy, confocal microscopy, flow cytometry (cell membrane staining and/or intracellular staining) or cells lysates are made and analyzed on HPLC and mass-spectrometry. NF-κB transfected (reporter gene assay) cells and gene array technology can be used to determine the regulatory effects of peptides.

HPLC and mass-spectrometry analysis: Purified NF-κB subunit or cytosolic/nuclear extract is incubated in the absence or presence of (regulatory) peptide is diluted (2:1) with 8 N guanidinium chloride and 0.1% trifluoroacetic acid, injected into a reverse-phase HPLC column (Vydac C18) equilibrated with solvent A (0.1% trifluoroacetic acid), and eluted with a gradient of 0 to 100% eluant B (90% acetonitrile in solvent A). Factions containing NF-κB subunit are pooled and concentrated. Fractions are then dissolved in appropriate volume and could be analyzed on mass-spectrometry.

Acute phase proteins (APP) or acute phase reactants (APR) is the generic name given to a group of approximately 30 different biochemically and functionally unrelated proteins. The levels of acute phase proteins in the serum are either increased (positive acute phase reactants) or reduced (negative acute phase reactants) approximately 90 minutes after the onset of an inflammatory reaction. The more important acute phase proteins are usually lycoproteins. Exceptions are C-reactive protein (CRP) and serum amyloid A protein (SAA).

Acute phase proteins of inflammation and their function include:

alpha-1 acid glycoprotein (interaction with collagen, promotion of fibroblast growth, binding of certain steroids), alpha-1 antichymotrypsinogen (protease inhibitor), alpha-1 antitrypsin (protease inhibitor, resolution of emphysema), alpha-2 antiplasmin (modulation of coagulation cascade), alpha-2-Macroglobulin (inhibitor of several serum proteases and other functions), antithrombin-3 (modulation of coagulation cascade), C1 (inhibitor negative control of complement cascade), C2, C4, C4 binding protein, C5 and C9 as complement component, C-reactive protein (binding to membrane phosphorylcholine, complement activation and opsonization, interaction with T-cells and B-cells), Ceruloplasmin (copper transport protein, reactive oxygen scavenger), Factor VIII (clotting formation of fibrin matrix for repair), Factor-B complement component), Ferritin (iron transport protein), Fibrinogen (clotting formation of fibrin matrix for repair), Fibronectin (fibrin clot formation), Haptoglobin (hemoglobin scavenger), Heme oxygenase (heme degradation), Hemopexin (heme binding and transport protein), Heparin cofactor-2 (proteinase inhibitor), Kallikreins (vascular permeability and dilatation), LPS binding protein (macrophage cell activation), Manganese superoxide dismutase (copper zinc binding protein, formation of reactive oxygen species), Mannose-binding protein (serum lectin), Plasminogen (proteolytic activation of complement, clotting, fibrinolysis), Plasminogen activator inhibitor-1 (protease inhibitor), Prothrombin (clotting formation of fibrin matrix for repair), Serum amyloid A (cholesterol and HDL scavenger), Serum amyloid-P (formation of IgG immune complexes), von Willebrand factor (coagulation protein), IL1ra (IL1 receptor antagonist).

IL1ra (IL1 receptor antagonist) has been shown recently to be regulated by various pro-inflammatory cytokines in the same way as other acute phase proteins.

Acute phase proteins are synthesized predominantly in the liver with each hepatocyte possessing the capacity to produce the entire spectrum of these proteins. Following stimulation of single hepatocytes within individual lobules one observes a stimulation of further hepatocytes and this process continues until almost all hepatocytes produce these proteins and release them into the circulation. The various acute phase proteins differ markedly in the rise or decline of their plasma levels and also in their final concentrations. Nevertheless, acute phase responses generate a characteristic serum protein profile. Levels of elevated expression can differ widely from species to species and some proteins that function as an acute phase protein in one species may not be an acute phase protein in another species.

Acute phase proteins regulate immune responses, function as mediators and inhibitors of inflammation, act as transport proteins for products generated during the inflammatory process (the heme-binding protein hemopexin, and Haptoglobin), and/or play an active role in tissue repair and remodeling (Wound healing). Van Molle et al. suggest that at least some acute phase proteins might constitute an inducible system of factors protecting against cell death by apoptosis. They observe that alpha1-acid glycoprotein and alpha1-antitrypsin activate the major executioners of apoptosis, caspase-3 and caspase-7.

Some of the acute phase proteins behave like cytokines. C-reactive protein, for example, activates macrophages (MAF, macrophage activating factor) while some other acute phase proteins influence the chemotactic behavior of cells (MIF, migration inhibition factor). Some acute phase proteins possess antiproteolytic activity and presumably block the migration of cells into the lumen of blood vessels thus helping to prevent the establishment of a generalized systemic Inflammation. A failure to control these processes, i.e., an uncontrolled acute phase reaction, eventually has severe pathological consequences such as Systemic inflammatory response syndrome.

The elevated serum concentrations of certain acute phase proteins are of diagnostic relevance and also of prognostic value. Their measurement, for example, allows inflammatory processes to be distinguished from functional disturbances with similar or identical clinical pictures. Under normal circumstances an acute phase response is not observed with functional disturbances that are not the result of an inflammatory process, thereby allowing the differentiation between failure of function and organic disease.

Some acute phase reactions are observed also in chronic disorders such as rheumatoid arthritis and chronic infections. There are many diseases in which the rise in the synthesis of acute phase proteins parallels the degree and progression of the inflammatory processes.

The coordinated expression of many acute phase proteins as a direct consequence of the activities of several cytokines can be explained, at least in part, by the fact that the regulatory sequences of the genes encoding these acute phase proteins contain so-called cytokine response elements (for example IL6RE as an IL6-specific element). These elements are recognized specifically by transcription factors that mediate the activity of these genes in a cell- and/or tissue-specific manner.

The major inducers of acute phase proteins are IL1, IL6, and TNF. The two mediators IL1 and IL6 have been used to classify acute phase proteins into two subgroups. Type-1 acute phase proteins are those that require the synergistic action of IL6 and IL1 for maximum synthesis. Examples of Type-1 proteins are C-reactive protein, serum amyloid A and alpha-1 acid glycoprotein. Type-2 acute phase proteins are those that require IL6 only for maximal induction. Examples of Type-2 proteins are fibrinogen chains, haptoglobin, and alpha-2-Macroglobulin. Expression of genes encoding Type-2 acute phase proteins is suppressed rather than being enhanced frequently by IL1(Ramadori et al.; Fey et al.). Additive, synergistic, co-operative, and antagonistic effects between cytokines and other mediator substances influencing the expression of acute phase proteins do occur and have been observed in almost all combinations. Many cytokines also show differential effects, inducing the synthesis of one or two acute phase proteins but not others. For example, Activin A induces a subset of acute phase proteins in HepG2 cells. Bacterial lipopolysaccharides and several cytokines (mainly IL1, IL6 and TNF but also LIF, CNTF, oncostatin M, IL11, and cardiotrophin-1) are involved in the induction of SAA synthesis and some of these cytokines act synergistically.

IL1 and also IFN-gamma reduce some of the effects of IL6. Some of the effects of IL2 and IL6 are antagonized by TGF-beta. The combined action of two or even more cytokines may produce effects that no factor on its own would be able to achieve. In cultured HepG2 hepatoma cells IL1, IL6, TNF-alpha and TGF-beta induce the synthesis of antichymotrypsin and at the same time repress the synthesis of albumin and AFP (alpha-Fetoprotein). The synthesis of fibrinogen is induced by IL6 and this effect is, in turn, suppressed by IL1-alpha, TNF-alpha or TGF-beta-1. The increased synthesis of Haptoglobin mediated by IL6 is suppressed by TNF-alpha. Insulin inhibits the synthesis of some negative acute phase proteins (pre-albumin, Transferrin, and fibrinogen, in HepG2 hepatoma cells. For further information see also: Acute phase reaction, Inflammation, and Systemic inflammatory response syndrome.

The fact that different patterns of cytokines are involved in systemic and localized tissue damage is supported by observations with knock-out mice for IL1 and IL6. Inflammatory acute phase response after tissue damage or infection is severely compromised in IL6 knock-out mice, but only moderately affected after challenge with bacterial lipopolysaccharides. Also, in the absence of IL6, the induction of acute phase proteins is dramatically reduced in response to turpentine injections but those parameters are altered to the same extent both in wild-type and IL6-deficient mice following injection of bacterial lipopolysaccharides. These mice, however, produce three times more TNF-alpha than wild-type controls. Also, a normal acute phase reaction was observed to both turpentine and bacterial lipopolysaccharides in TNF-beta knock-out mice. There is, however, a striking absence of elevated major acute phase proteins, SAP and SAA, in mice deficient in TNF-beta and IL6.

### Regulation of Acute Phase Reactions and Synthesis of Acute Phase Proteins

Inflammatory cytokines such as IL6, IL1, TNF, and others such as TGF, IFN, and LIF are produced by inflammatory cells. They induce local and systemic reactions. Among other things these mediators are involved in cell activation of leukocytes, fibroblasts, endothelial cells, and smooth muscle cells, inducing the synthesis of further cytokines. These mediators also have direct actions in hepatocytes of the liver. Activities are enhanced indirectly by activation of the pituitary/adrenal gland axis which involves synthesis of ACTH and subsequent production of cortisol. Cortisol can enhance expression of IL6 receptors in liver cells and thus promotes IL6 mediated synthesis of acute phase proteins.

Negative regulatory loops can involve inhibition of synthesis of IL6, IL1, and TNF by cortisol and inhibition of the synthesis of IL1 and TNF in monocytes by IL6. Of all mediators participating in the induction and regulation of acute phase protein synthesis IL6 appears to induce the broadest spectrum of acute phase proteins whereas IL1 and TNF only induce the synthesis of subsets of these proteins.

Almost all cytokines are pleiotropic effectors showing multiple biological activities. In addition, multiple cytokines often have overlapping activities and a single cell frequently interacts with multiple cytokines with seemingly identical responses (cross-talk). One of the consequences of this functional overlap is the observation that one factor may frequently functionally replace another factor altogether or at least partially compensate for the lack of another factor.

Many cytokines show stimulating or inhibitory activities and may synergize or antagonize also the actions of other factors. A single cytokine may elicit reactions also under certain circumstances which are the reverse of those shown under other circumstances. The type, the duration, and also the extent of cellular activities induced by a particular cytokine can be influenced considerably by the micro-environment of a cell, depending, for example, on the growth state of the cells (sparse or confluent), the type of neighboring cells, cytokine concentrations, the combination of other cytokines present at the same time, and even on the temporal sequence of several cytokines acting on the same cell. Under such circumstances combinatorial effects thus allow a single cytokine to transmit diverse signals to different subsets of cells.

The fact that every cell type may have different responses to the same growth factor can be explained, at least in part, by different spectrums of genes expressed in these cells and the availability and levels of various transcription factors that drive Gene expression. The responses elicited by cytokines are therefore contextual and the “informational content,” i.e., the intrinsic activities of a given cytokine may vary with conditions. Although a variety of cytokines are known to share at least some biological effects the observations that single cells usually show different patterns of gene expression in response to different cytokines can be taken as evidence for the existence of cytokine receptor-specific signal transduction pathways. Shared and different transcriptional activators that transduce a signal from a cytokine receptor to a transcription regulatory element of DNA are involved in these processes (for example, STAT proteins, Janus kinases, IRS).

It has been observed, for example, that bFGF is a strong mitogen for fibroblasts at low concentrations and a chemoattractant at high concentrations. bFGF has been shown also to be a biphasic regulator of human hepatoblastoma-derived HepG2 cells, depending upon concentration. The interferon IFN-gamma can stimulate the proliferation of B-cells pre-stimulated with Anti-IgM, and inhibits the activities of the same cells induced by IL4. On the other hand, IL4 activates B-cells and promotes their proliferation while inhibiting the effects induced by IL2 in the same cells. The activity of at least two cytokines (IL1-alpha and IL1-beta) is regulated by an endogenous receptor antagonist, the IL1 receptor antagonist (IL1ra). Several cytokines, including TNF, IFN-gamma, IL2 and IL4, are inhibited by soluble receptors. Several cytokines, including IL10 and TGF-beta, act to inhibit other cytokines.

In this study we demonstrate that LQGV (SEQ ID NO:1), AQGV (SEQ ID NO:2) and LAGV (SEQ ID NO:10), administrated after the induction of hemorrhagic shock in rats, significantly reduced TNF-α and IL-6 plasma levels, which is associated with reduced TNF-α and IL-6 mRNA transcript levels in the liver. This indicates that LQGV (SEQ ID NO:1), AQGV (SEQ ID NO:2), LAGV (SEQ ID NO:10) may have therapeutic potential with beneficial effects on systemic inflammation, thereby reducing organ integrity/function, which is associated with shock and SIRS often seen with severe burns patients.

### Materials and Methods

Adult Male specific pathogen-free Wistar rats (Harlan CPB, Zeist, NL), weighing 350-400 g were used after a minimum seven-day acclimation period. The animals were housed under barrier conditions and kept at 25° C. with a twelve-hour light/dark cycle. Rats were allowed free access to water and chow (−). All procedures were performed in accordance with the Principles of Laboratory Animal Care (NIH publication No. 86-23, revised 1985) under a protocol approved by the Committee on Animal Research of the Erasmus University (protocol EUR 365).

The rats were fasted overnight but were allowed free access to water before the experiment. Subsequent to endotracheal intubation the rats were mechanically ventilated with an isoflurane (−) N₂O/O₂ mixture at 60 breaths/minute. Body temperature was continuously maintained at 37.5° C. by placing the animals on a thermo controlled “half-pipe” (UNO, NL). Polyethylene tubes (PE-50, Becton Dickinson; St. Michielsgestel, NL) were flushed with heparin and placed via the right carotid artery in the aorta and in the right internal jugular vein. The animals received no heparin before or during the experiment.

Mean arterial pressures (MAP) was measured using transducers (Becton Dickinson) that were connected in line to an electronic recorder (Hewlett Packard, 78354-A Germany) for electronically calculated mean pressures and continuous measurement of the animal's blood pressure. Under semi sterile conditions a median laparotomy was performed and ultrasonic perivascular flow probes (Transonic Systems Inc, Maastricht, NL) were placed on the common hepatic artery and the portal vein. A supra pubic catheter was placed to monitor the urine production during and after resuscitation.

After an acclimatization period of 20 minutes, the rats were randomized into the following five groups:

Hemorrhagic shock group were bled within ten minutes to a mean arterial pressure (MAP) of 40 mmHg and maintained at this level for 60 minutes by withdrawing or re-infusing shed blood as needed. Thereafter, the animals were resuscitated with plus minus four times the volume of the withdrawn blood over 30 minutes with a 0.9% NaCl solution.

The hemorrhagic shock group+peptide A (LAGV (SEQ ID NO:10); one-letter amino acid code) underwent the same procedure as the hemorrhagic shock group but received a single bolus injection of 5 mg/kg peptide A intravenously 30 minutes after the induction of shock.

The hemorrhagic shock group+peptide B (AQGV (SEQ ID NO:2)) underwent the same procedure as the hemorrhagic shock group and received a single bolus injection of 5 mg/kg peptide B intravenously, 30 minutes after the induction of shock.

The hemorrhagic shock group+peptide C (LQGV (SEQ ID NO: 1)) underwent the same procedure as the hemorrhagic shock group and received a single bolus injection of 5 mg/kg peptide C intravenously, 30 minutes after the induction of shock.

Sham group underwent the same procedure as the hemorrhagic shock group without performing the hemorrhage or administration of any kind of peptides.

The hepatic arterial blood flow (QHA) and hepatic portal venous blood flow (QVP) were measured with transit time ultrasonic perivascular flow probes, connected to an ultrasonic meter (T201; Transonic Systems, Inc., Maastricht, NL). Systemic and hepatic hemodynamics were continuously measured. At regular time points arterial blood samples were taken. The animals were euthanized by withdrawal of arterial blood via the carotid artery.

### Blood, Tissue, and Cell Harvesting Procedure

Plasma collection and storage: Whole arterial blood was obtained at −15, 30, 60, 90, 120, 150 and 180 minutes after induction of shock via the right carotid artery and collected in duplo. 0.2 ml was placed in tubes (Eppendorf EDTA KE/1.3) to be assayed in the coulter counter (−). 0.5 ml was placed in Minicollect tubes (Bio-one, Greiner) centrifuged for five minutes, immediately frozen, and stored at −80° C., until assayed. All assays were corrected for the hematocrit.

Measurement of cytokines (still in progress): The levels of IL-6, and IL-10 in the serum were determined by an ELISA (R&D Systems Europe Ltd) according to the manufacturer's instructions.

Histology (still in progress): The alterations in lung, liver, sigmoid and small bowel morphology were examined in sham-operated animals, in animals after trauma-hemorrhage and in animals after trauma-hemorrhage treated with peptide A, B or C. All tissues were collected in duplo. One part was harvested and fixed in formalin (Sigma) and later embedded in paraffin. The other part was placed in tubes (NUNC Cryo Tube™ Vials), quick frozen in liquid nitrogen and stored at −80° C. until assayed.

### Results

Mean Arterial Pressure: MAP dropped in all shock groups significant during the shock phase compared to the control group.

Hematocrit: The hematocrit following trauma-hemorrhage was similar in the different peptide A, B and C treated and non-treated groups. During the shock phase there was a difference of hematocrit in the control group in comparison with the other groups. From the resuscitation phase (90 minutes) there was no significant difference in hematocrit among the control, trauma-hemorrhage, and peptide groups.

Leukocyte Recruitment: During trauma-hemorrhage the leukocytes dropped from 100% at TO in all groups to a minimum of 40.0±11.9%, 42.0±8.7%, 47.3±12.4%, 38.2±7.4% in respectively the non-treated, peptide A treated, peptide B treated and peptide C treated group because of leukocyte accumulation in the splanchnic microcirculation. There was a significant difference in leukocyte concentration between all treated and non-treated trauma-hemorrhage groups, and the control group during the shock phase. No significant difference was noticed between the peptide A, B or C treated animals and the non-treated animals.

Blood Concentrations of Macrophages and Granulocytes: At 180 minutes after the onset of trauma-hemorrhage, concentrations of circulating macrophages (M_{φ}) and granulocytes were significant lower in the peptide B and C treated animals compared with the corresponding experimental group. Blood levels of circulating M_{φ} and granulocytes were 5,556±1,698 10⁹/l in sham-operated animals whereas blood levels were 6,329±1,965 10⁹/l after trauma-hemorrhage, and decreased by 29.9% after administration of peptide B (4,432±0.736 10⁹/l) and 39.2% after administration of peptide C (3,846±0.636 10⁹/l) compared with concentrations after trauma-hemorrhage.

Arterial Hepatic Blood Flow: There was a decrease in the arterial hepatic blood flow in the shock group (18.3±14.3%) and in the peptide A (21.3±9.1%), B (18.1+9.0%) and C (21.2±8.6%) group during the shock period compared with the control group (102.6±23.5%). An increase in blood flow was observed during the reperfusion in the hepatic artery of the shock group (128.9±75.4%) compared with control animals (83.7±24.2%) and the animals treated with peptide B (78.4±28.3%).

Trauma-hemorrhage results in hypoxic stress owing to the absolute reduction in circulating blood volume. In contrast, sepsis is an inflammatory state mainly mediated by bacterial products. It is interesting that these divergent insults reveal similar pathophysiologic alterations in terms of the splanchnic circulation.

Hemorrhagic shock significantly increases leukocyte accumulation in the splanchnic microcirculation owing to the up-regulation of P selectin. The expression of intercellular adhesion molecule within the intestinal muscular vasculature after hemorrhagic shock promotes the local recruitment of leukocytes, and this inflammatory response is accompanied by subsequent impairment of intestinal function.

The adhesion and extravasation of neutrophils not only contribute to the inflammatory response in the splanchnic tissue bed but also induce intestinal microcirculatory failure and dysfunction after severe stress. This is mediated by the induced expression of adhesion molecules, such as selectins and endothelial cell adhesion molecules, on the surface of neutrophils and endothelial cells.

In the shock experiments, leukocyte concentration significant decreases during hemorrhagic shock compared to the control animals. However a single dose of peptide B or C administered during resuscitation, decreased concentrations of circulating macrophages and granulocytes 120 minutes after the onset of hemorrhagic shock compared to the non-treated animals.

Because some female sex hormones effectively protect the organs from circulatory failure after various adverse circulatory conditions, numerous studies have been performed to clarify the molecular mechanism of for example estradiol action with regard to tissue circulation. In this study, a single dose of peptide was administered following trauma-hemorrhage and various parameters were measured at three hours following the induction of sepsis. Treatment with peptides improved or restored immune functional parameters and cardiovascular functions. Therefore, our results show that administration of short oligopeptides (NMPFs) is beneficial in the treatment of critically ill trauma victims experiencing hemorrhagic shock.

### Example 2

BACKGROUND: Hemorrhagic shock followed by resuscitation induces a massive pro-inflammatory response, which may culminate into severe inflammatory response syndrome, multiple organ failure and finally death. Treatments aimed at inhibiting the effects of pro-inflammatory cytokines are only effective when initiated before the onset of hemorrhagic shock, which severely limits their clinical application.

AIM: We investigated whether the administration of synthetic hCG-related oligopeptides (LQGV (SEQ ID NO:1), AQGV (SEQ ID NO:2), LAGV (SEQ ID NO:10)) 30 minutes after induction of hemorrhagic shock reduced the inflammatory response.

METHODS: Rats were bled to 50% of baseline mean arterial pressure and one hour later resuscitated by autologous blood transfusion. Thirty minutes after onset of hemorrhagic shock, experimental groups received either one of the synthetic hCG-related oligopeptides (LQGV (SEQ ID NO:1), AQGV (SEQ ID NO:2), LAGV (SEQ ID NO:10)) or 0.9% NaCl solution. TNF-α and IL-6 plasma levels were determined at fixed time points before and after onset of hemorrhagic shock. Liver, lungs, ileum and sigmoid mRNA levels for TNF-α, IL-6 and ICAM-1 were determined 180 minutes after onset of hemorrhage.

RESULTS: Treatment with either one of the three hCG-related oligopeptides (LQGV (SEQ ID NO:1), AQGV (SEQ ID NO:2), LAGV (SEQ ID NO:10)) efficiently reduced TNF-α and IL-6 plasma levels as well as TNF-α and IL-6 mRNA transcript levels in the liver.

CONCLUSION: Considering these powerful effects of hCG-related oligopeptides during severe hemorrhagic shock, they may have therapeutic potential with beneficial effects on the hyper inflammation, thereby reducing the late life threatening tissue- and organ-damage that is associated with severe hemorrhagic shock which can be a consequence of surgery.

INTRODUCTION: In hemorrhagic shock there is massive blood loss, which cannot be compensated by the body without treatment. The primary treatment of hemorrhagic shock is to control bleeding and restore intravascular volume to improve tissue perfusion. This treatment induces an inflammatory response, which may culminate into a severe inflammatory response and finally multiple organ dysfunction syndrome (MODS).^{[1, 2, 3]} In addition, approximately 40% of patients develop sepsis as a result of trauma-hemorrhage.^{[3]} Sepsis and MODS are the leading causes of death in critically ill patients on the intensive care unit all over the world with mortality rates of about 50%.^{[4, 5]}

The severe inflammatory response due to trauma-hemorrhage is characterized by increased expression of adhesion molecules, such as intracellular adhesion molecule-1 (ICAM-1) and vascular cell adhesion molecule-1 (VCAM-1), on sinusoidal endothelial cells and hepatocytes. Furthermore, increased levels of pro-inflammatory cytokines are found systemically and locally in liver, lungs and intestine. [6, 7, 8, 9] The pro-inflammatory cytokines produced are in particular tumor necrosis factor alpha (TNF-α), interleukin (IL)-1 and IL-6.^{[10, 11, 12]} These cytokines affect organ integrity/function directly, but also indirectly through secondary mediators, such as nitric oxide, thromboxanes, leukotrienes, platelet-activating factor, prostaglandins, and complement.^{[13, 14]} TNF-α also causes the release of tissue-factor by endothelial cells leading to fibrin deposition and disseminated intravascular coagulation.^{[15, 16]} Cells within the liver, mainly Kupffer cells, but also hepatocytes and sinusoidal endothelial cells, are considered as the main producers of these pro-inflammatory cytokines during hemorrhagic shock.^{[17]}

The last decade, researchers have focused on the modulation of the systemic inflammatory responses with therapeutic agents aiming at neutralizing the activity of cytokines, especially TNF-α^{[18]} Other researchers used therapeutic agents aiming at the inhibition of TNF-α production^{[19]} However, most of these therapeutic agents must be administered before the onset of hemorrhagic shock to achieve a therapeutic effect.^{[19}] Clearly, this is almost impossible in a clinical trauma-hemorrhage setting. Therefore, therapies initiated after the onset of severe trauma-hemorrhage and aiming at reducing the production of pro-inflammatory cytokine are more relevant to prevent the events leading to MODS.

During pregnancy, the maternal immune system tolerates the fetus by reducing the cell-mediated immune response while retaining normal humoral immunity.^{[20]} Also, clinical symptoms of cell-mediated autoimmune diseases regress in many patients during pregnancy.^{[20]} The hormone human chorionic gonadotropin (hCG) is mainly secreted by placental syncytiocytotrophoblasts during pregnancy and has been shown to be immunoregulatory.^{[21, 22, 23]} The β-subunit of hCG is degraded by specific proteolytic enzymes.^{[24]} This can lead to the release of several oligopeptides consisting of four to seven amino acids which, because of their role in regulation of physiological processes, are considered regulatory.^{[25]} We successfully demonstrated that synthetic hCG-related oligopeptides can inhibit the acute inflammatory response, disease severity, and mortality in high-dose lipopolysaccharide induced systemic inflammatory response syndrome.^{[26]} Considering these powerful regulating effects of synthetic hCG-related oligopeptides on inflammation, we hypothesized that the administration of such regulatory oligopeptides after severe trauma-hemorrhage could inhibit the massive inflammatory response, associated with this condition. To this end, we used LQGV (SEQ ID NO:1), which is part of the primary structure of loop two of the β-subunit of hCG, and two alanine replacement variants, namely AQGV (SEQ ID NO:2) and LAGV (SEQ ID NO:10).

In this study we demonstrate that LQGV (SEQ ID NO:1), AQGV (SEQ ID NO:2), and LAGV (SEQ ID NO:10), administrated after the induction of hemorrhagic shock in rats, significantly reduced TNF-α and IL-6 plasma levels, which is associated with reduced TNF-α and IL-6 mRNA transcript levels in the liver. This indicates that LQGV (SEQ ID NO:1), AQGV (SEQ ID NO:2), LAGV (SEQ ID NO:10) may have therapeutic potential with beneficial effects on systemic inflammation, thereby reducing organ integrity/function, which is associated with severe hemorrhagic shock.

### Materials and Methods

### Animals

Adult male specific pathogen-free Wistar rats (Harlan CPB, Zeist, NL), weighing 350-400 g were used. Animals were housed under barrier conditions at −25° C. with a twelve-hour light/dark cycle, and were allowed food and water ad libitum. The experimental protocol was approved by the Animal Experiment Committee under the Dutch Experiments on Animals Act and adhered to the rules laid down in this national law that serves the implementation of “Guidelines on the protection of experimental animals” by the Council of Europe (1986), Directive 86/609/EC.

hCG-related synthetic oligopeptides: The hCG-related oligopeptides (LQGV (SEQ ID NO:1), AQGV (SEQ ID NO:2) and LAGV (SEQ ID NO:10)) were synthesized by Ansynth Service B. V. (Roosendaal, NL) and dissolved in 0.9% NaCl at a concentration of 10 mg/ml.

Surgical procedures: Rats were food deprived overnight before the experiment, but were allowed water ad libitum. Rats were anesthetized using a mixture of N₂O/O₂ isoflurane (Pharmachemie B.V., Haarlem, NL). Body temperature was continuously maintained at 37.5° C. by placing the rats on a thermo controlled “half-pipe” (UNO, Rotterdam, NL). Endotracheal intubation was performed, and rats were ventilated at 60 breaths per minute with a mixture of N₂O/O₂ 2% isoflurane. Polyethylene tubes (PE-50, Becton Dickinson; St. Michielsgestel, NL) were flushed with heparin and placed via the right carotid artery in the aorta and in the right internal jugular vein. The rats received no heparin before or during the experiment.

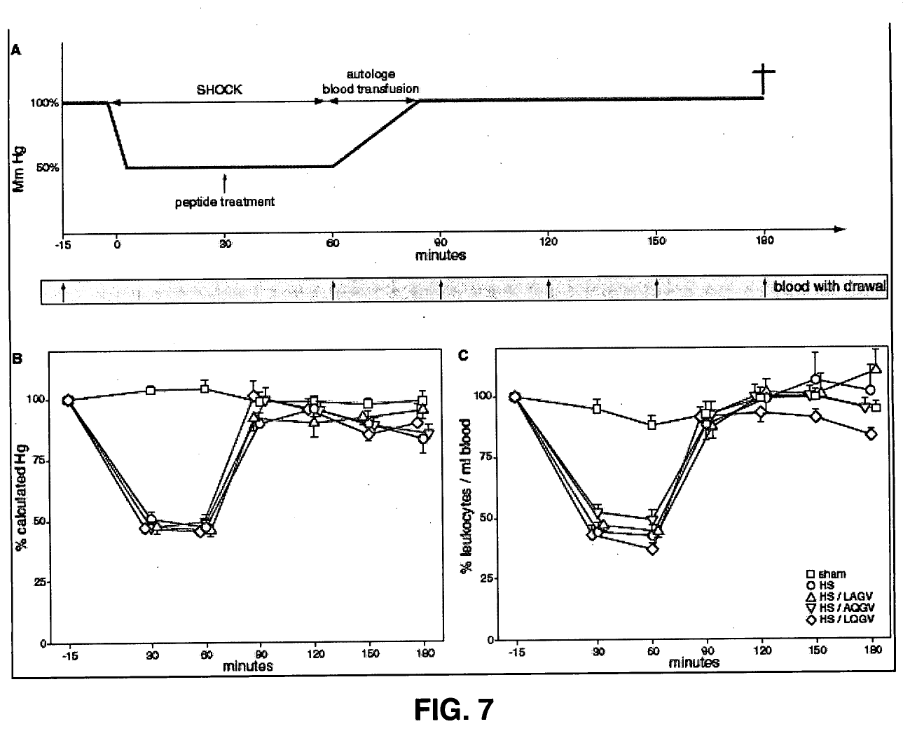

Experimental procedures: After an acclimatization period of 15 minutes, the rats were randomized into five different groups: 1) sham, 2) hemorrhagic shock (HS), 3) hemorrhagic shock with LQGV (SEQ ID NO:1) treatment (HS/LQGV), 4) hemorrhagic shock with AQGV (SEQ ID NO:2) treatment (HS/AQGV (SEQ ID NO:2)) and 5) hemorrhagic shock with LAGV (SEQ ID NO:10) treatment (HS/LAGV (SEQ ID NO:10)). Hemorrhagic shock was induced by blood withdrawal, reducing the circulating blood volume until a mean arterial pressure (MAP) of 50% of normal mmHg was reached. This level of hypotension was maintained for 60 minutes. After 30 minutes, rats received either a single bolus injection of 10 mg/kg LQGV (SEQ ID NO:1), AQGV (SEQ ID NO:2), LAGV (SEQ ID NO:10) or 0.9% NaCl solution. The peptides and dosage were based on previous studies, in which we performed dose-escalation experiments (manuscript in preparation). Sixty minutes after induction of hemorrhagic shock, rats were resuscitated by autologous blood transfusion over a period of 30 minutes and monitored for another 120 minutes after which they were sacrificed (FIG. 7A). Sham animals underwent the same surgical procedure as the hemorrhagic shock animals, but without performing hemorrhage and administration of peptides.

Plasma collection and storage: Arterial blood was obtained 15 minutes before and 30, 60, 90, 120, 150 and 180 minutes after onset of hemorrhage (FIG. 7A). After blood withdrawal, leukocyte numbers were determined using a coulter counter (Beckman Coulter, Mijdrecht, NL) and corrected for the hematocrit. Approximately, 0.3 ml of blood was placed into mini collect tubes (Greiner, Bio-one, Alphen a/d Rijn, NL), plasma was obtained by centrifugation (1500 rpm; five minutes), immediately frozen, and stored at −80° C., until assayed.

Measurements of Mean arterial pressure: During the experiments, mean arterial pressure (MAP) was continuously measured using transducers (Becton Dickinson) that were connected in line to an electronic recorder (Hewlett Packard, 78354-A, Germany).

Tissue collection and storage: Liver, lungs, ileum and sigmoid were surgically removed at the end of the experiment, snap-frozen, and stored at −80° C., until assayed.

Measurement of cytokines: TNF-α and IL-6 plasma levels were determined by ELISA (R&D Systems Europe Ltd, Abingdon, UK), according to the manufacturer's instructions.

Evaluation of mRNA levels by real-time quantitative (RQ)-PCR: RNA was isolated using a QIAGEN kit (QIAGEN, Hilden, Germany), according to the manufacturer's instructions. TNF-α, IL-6 and ICAM-1 transcripts were determined by RQ-PCR using an Applied Biosystems 7700 PCR machine (Foster City, Calif., USA) as described previously.^{[27]} TNF-α, IL-6 and ICAM-1 expression was quantified by normalization against GAPDH. Primer probe combinations used are listed in Table 1.

Statistical analysis: Statistical analysis was performed using SPSS version 11 software (SPSS Inc., Chicago, Ill.). Inter group differences were analyzed with Kruskal-Wallis statistical test. If Kruskal-Wallis statistical testing resulted in a p<0.05, a Dunn's Multiple Comparison test was performed and p<0.05 was considered statistically significant.

### Results

Induction of hemorrhagic shock: Lowering the MAP to 50% of normal induced hemorrhagic shock, which was successfully maintained for 60 minutes in all four experimental groups (FIG. 7B). No change in MAP was observed in sham treated rats (FIG. 7B). A decrease in the percentage of blood leukocytes was observed in all four experimental groups after blood withdrawal (FIG. 7C). Sixty minutes after hemorrhagic shock, rats were resuscitated with there own blood to induce organ reperfusion, which was associated with a normalization of leukocyte level (FIG. 7C).

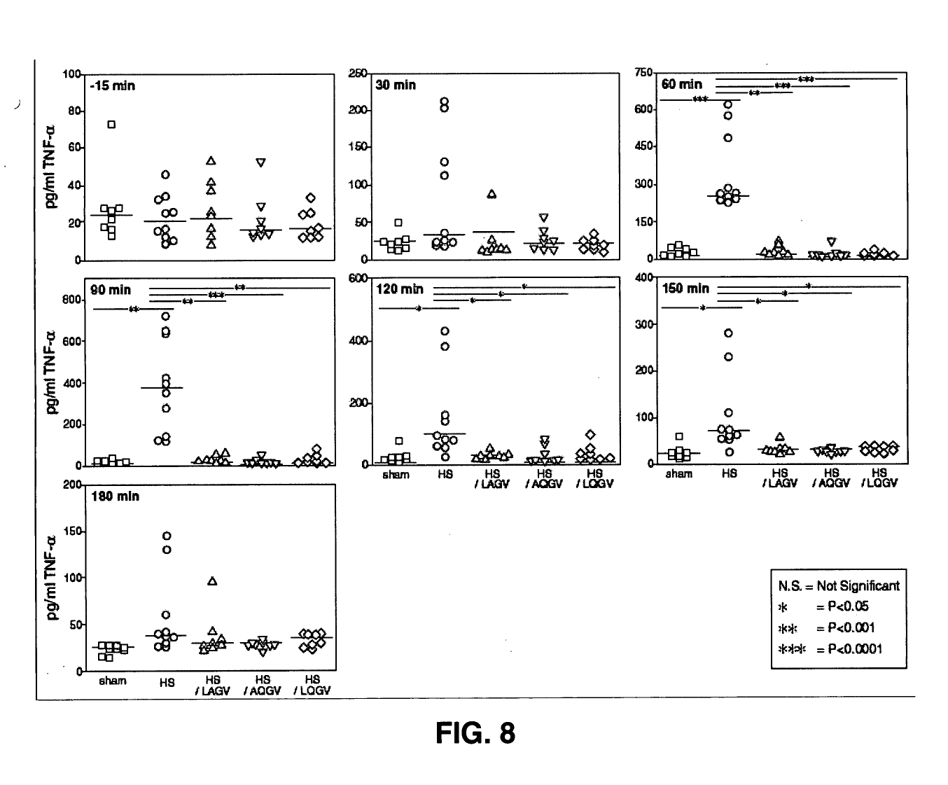

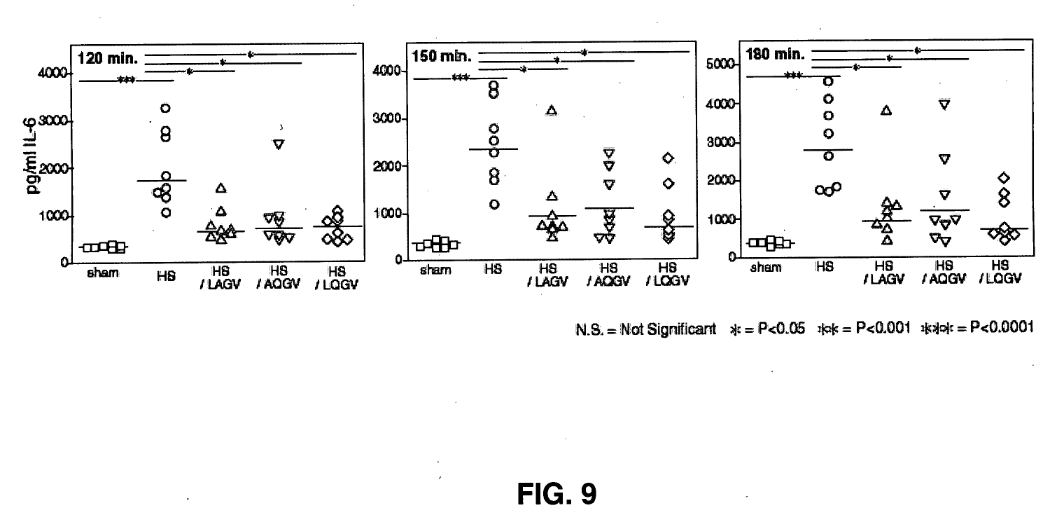

Oligopeptide treatment reduces pro-inflammatory cytokine plasma levels: The therapeutic capacity of three synthetic oligopeptides (LQGV (SEQ ID NO: 1), AQGV (SEQ ID NO:2), LAGV (SEQ ID NO: 10)) related to the primary structure of loop two of the β-subunit of hCG was evaluated in a rat hemorrhagic shock model. Before induction of hemorrhage, TNF-α plasma levels were comparable in all five groups (˜15-24 μg/ml) (FIG. 8). In the HS group, TNF-α levels started to increase thirty minutes after induction of hemorrhagic shock and were significantly increased after sixty minutes, as compared to the sham group (264 pg/ml vs 24 pg/ml, respectively; p<0.01). TNF-α levels reached a maximum of 374 pg/ml after 90 minutes in the HS group, after which levels declined again but always remaining increased compared to the sham group (FIG. 8). In contrast, none of the oligopeptide-treated HS groups (HS/LQGV (SEQ ID NO:1), HS/AQGV (SEQ ID NO:2), HS/LAGV (SEQ ID NO:10)) showed an increase in plasma TNF-α levels during the experiment (FIG. 8). IL-6 levels are known to increase at a later time-point than TNF-α after severe hemorrhagic shock.^{[11, 12]} Therefore, we determined IL-6 levels in blood samples collected 120, 150 and 180 minutes after the onset of hemorrhagic shock. In the HS group, IL-6 plasma levels were significantly increased as compared to sham group at 120 minutes (1704 pg/ml vs 338 pg/ml, respectively; p<0.001), at 150 minutes (2406 pg/ml vs 316 pg/ml, respectively; p<0.001) and at 180 minutes (2932 pg/ml vs 369 pg/ml, respectively; p<0.001) (FIG. 9). Although IL-6 levels tended to increase a little in the HS/oligopeptide treated rats as compared to sham treated rats, this never reached significance. Treatment with oligopeptides after hemorrhagic shock (HS/LQGV (SEQ ID NO:1), HS/AQGV (SEQ ID NO:2), HS/LAGV (SEQ ID NO:10)) resulted in a significant reduction of IL-6 plasma levels as compared to the non-treated hemorrhagic shock group (HS) (FIG. 9). These data demonstrate that treatment with a single dose of either LQGV (SEQ ID NO:1), AQGV (SEQ ID NO:2), or LAGV (SEQ ID NO:10) after induction of hemorrhagic shock results in a significant reduction of TNF-α and IL-6 plasma levels.

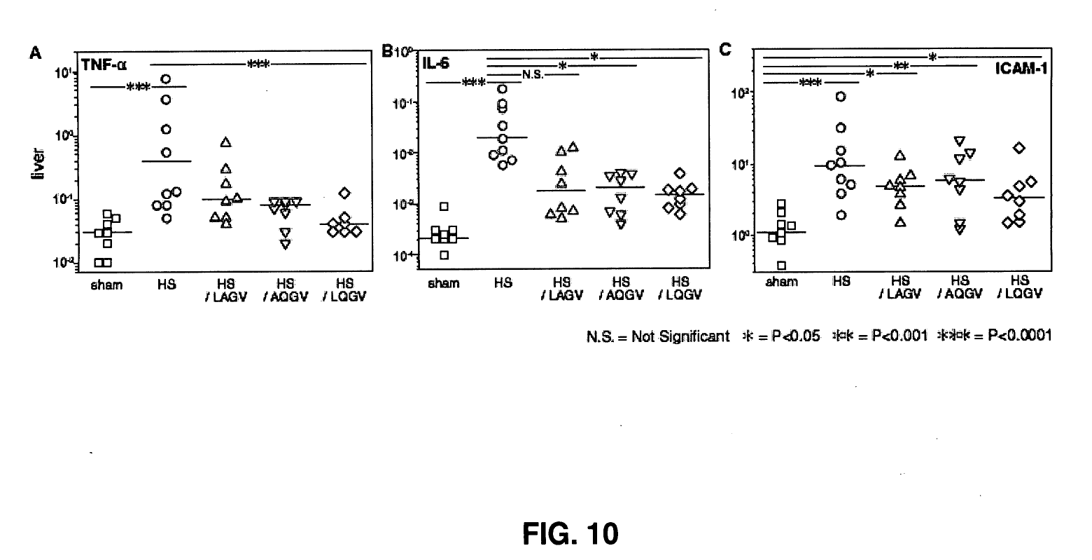

Oligopeptide treatment reduces TNF-α and IL-6 but not ICAM-1 mRNA levels in the liver: Because oligopeptide treatment clearly decreased the TNF-α and IL-6 plasma levels, we analyzed mRNA levels in liver, lungs, ileum and sigmoid tissues at 180 minutes after the onset of hemorrhagic shock. In the liver, TNF-α transcripts were significantly increased in the HS group as compared to the sham group. Oligopeptide treatment was associated with decreased TNF-α transcripts in the liver as compared to non-treated HS rats with only HS/LQGV showing a significant reduction as compared to HS (p<0.01; FIG. 10A).

In the HS group, IL-6 transcripts in the liver were increased ˜83 times as compared to the sham group (p<0.001; FIG. 10B). None of the oligopeptide treated groups showed an increase in IL-6 mRNA as compared to the sham treated group. LQGV (SEQ ID NO: 1) and AQGV (SEQ ID NO:2) treatment resulted in a significant reduction in IL-6 mRNA transcripts as compared to the HS group (p<0.05; FIG. 10B).

ICAM-1 transcript levels in the liver were significantly increased in the HS group as compared to the sham group (FIG. 10C). Oligopeptide treatment during hemorrhagic shock (HS/LQGV (SEQ ID NO:1), HS/AQGV (SEQ ID NO:2), HS/LAGV (SEQ ID NO:10)) did not affect the ICAM-1 transcript levels in the liver (FIG. 10C). In lungs, ileum and sigmoid tissue no significant differences could be detected between the various groups for TNF-α, IL-6 and ICAM-1 (data not shown). These data indicate that oligopeptide treatment following hemorrhagic shock decreases pro-inflammatory cytokine transcript levels in the liver but does not reduce ICAM-1 transcript levels.

### Discussion

In this study we used a rat model of hemorrhagic shock and demonstrated that administration of synthetic hCG-related oligopeptides (LQGV (SEQ ID NO:1), AQGV (SEQ ID NO:2), LAGV (SEQ ID NO:10)) 30 minutes after shock induction, efficiently reduces the pro-inflammatory cytokine levels associated with this condition. Our data demonstrate this to be the consequence of reduced expression of pro-inflammatory cytokine mRNA transcript levels in the liver.

Hemorrhagic shock is associated with an early adherence of leukocytes to the vascular endothelium as a result of a decreased blood volume.^{[28]} In our model a decrease in the percentage of leukocytes was detected in all four experimental groups after blood withdrawal. This indicates that all experimental groups experienced hemorrhagic induced shock. Resuscitation resulted in an increase of the percentages of leukocytes in the experimental groups.

Hemorrhagic shock followed by resuscitation induces a severe inflammatory response, which is characterized by an exaggerated production of early pro-inflammatory cytokines, such as TNF-α, IL1β, and subsequently IL-6.^{[10, 11, 12}] TNF-α is a key mediator of the innate immune system that is crucial for the generation of a local protective immune response against infectious or non-infectious agents.^{[9]} However, uncontrolled massive TNF-α production is lethal, as it spreads via the bloodstream into other organs thereby inducing tissue damage and promoting the production of secondary pro-inflammatory mediators, such as IL6.^{[10, 11]}

Despite improvement in treatment strategies, trauma-hemorrhage patients may still develop severe inflammatory response that leads too MODS and finally death. Experimental treatment strategies aimed at neutralizing bioactive cytokines, such as monoclonal antibodies against TNF-α, have been successfully applied in several inflammatory disorders, including Crohn's disease and Rheumatoid Arthritis.^{[29, 30]} However, clinical studies using monoclonal antibodies against TNF-α showed no clinical effect in trauma-patients.^{[}^{31]} It has been suggested that TNF-α neutralizing antibodies causes the accumulation of a large pool of TNF-α/anti-TNF-α pool, which act as a slow release reservoir that may lead to increased constant active TNF-α.^{[32]} Therefore, aiming at therapies that decrease the production of TNF-α and IL-6 may be more beneficial in limiting tissue damage and mortality rates in trauma-hemorrhage patients than neutralization of already produced cytokines.

In hemorrhagic shock, TNF-α is secreted within minutes after cellular stimulation, while production stops after three hours, and TNF-α plasma levels become almost undetectable.^{[9]} We demonstrate that hCG-related regulatory oligopeptides (LQGV (SEQ ID NO:1), AQGV (SEQ ID NO:2), LAGV (SEQ ID NO:10)), administered 30 minutes after the induction of hemorrhagic shock, significantly reduced TNF-α and IL-6 plasma levels. Whether the effect on IL-6 production is direct, or indirect due to reduced TNF-α plasma levels cannot be concluded from our data. Nevertheless, establishing a reduction of IL-6 is of clinical importance, because high IL-6 plasma levels correlate with poor outcome and decreased survival in patients with severe trauma and infection.^{[33, 34]} Cells within the liver, are considered as the main producers of pro-inflammatory cytokines during hemorrhagic shock.^{[17]} TNF-α and IL-6 transcript levels were significantly increased in the livers of the HS group. LQGV (SEQ ID NO:1), AQGV (SEQ ID NO:2) or LAGV (SEQ ID NO:10) treatment was associated with a reduction in TNF-α and IL-6 liver transcripts, which may be indicative of decreased transcriptional activation. Another important characteristic of endothelial cells and hepatocytes during hemorrhagic shock is increased expression of the adhesion molecule ICAM-1.^{[7, 8]} Our study confirms the increased ICAM-1 expression in the liver after hemorrhagic shock. However, LQGV (SEQ ID NO:1), AQGV (SEQ ID NO:2), or LAGV (SEQ ID NO:10) treatment did not result in reduced ICAM-1 expression. This could be due to the inability of hCG-related oligopeptides to interfere with induction of ICAM-1 transcription. In lungs, ileum and sigmoid, we detected no effect of hemorrhagic shock on the induction of TNF-α, IL-6 and ICAM-1 transcripts. This confirms that the liver is the first organ in which the inflammatory response is initiated after hemorrhagic shock and fluid resuscitation.^{[15, 31, 32]} In summary, a single administration of a synthetic hCG-related oligopeptide (LQGV (SEQ ID NO:1), AQGV (SEQ ID NO:2), LAGV (SEQ ID NO:10)) after the induction of severe trauma-hemorrhage reduces the subsequent pro-inflammatory response. These data suggest that these oligopeptides have therapeutic potential, in minimizing or preventing the late life threatening tissue- and organ-damage that is associated with SIRS seen with severe perioperative trauma.

### Example 2

### Treatment of Severe Skin Inflammations Such as Seen Treatment with the Drug Imiquimod (Aldara™)

To assess the activity of the various peptides with skin inflammations and tissue destruction seen for example with patients treated with the drug imiquimod (Aldara™) an animal model was developed in which these inflammations are generated via topical application of the inflammatory agent to the skin of experimental mice. For this purpose mice were treated with 4% or 5% imiquimod. Imiquimod is an immune response modifier used in the treatment of skin cancers. It is manufactured as a 4% or 5% cream (Aldara™). Imiquimod works by stimulating the immune system to release a number of chemicals called cytokines whereby it results in inflammation. The imiquimod is taken up by the so-called “toll-like receptor 7” on certain immune cells that are found in the outside part of the skin, the epidermis. Skin areas treated with imiquimod will be come inflamed. The effects include itching, burning, redness, ulceration (sores), scabbing, flaking and pain, typically an example wherein medical treatment (an iatrogenic event) comprises destruction or lysis of a cell or tissue of the subject. Particularly the mice treated with the 5% cream developed the intense inflammatory skin lesions sometimes seen with iatrogenic wounds in skin cancer patients.

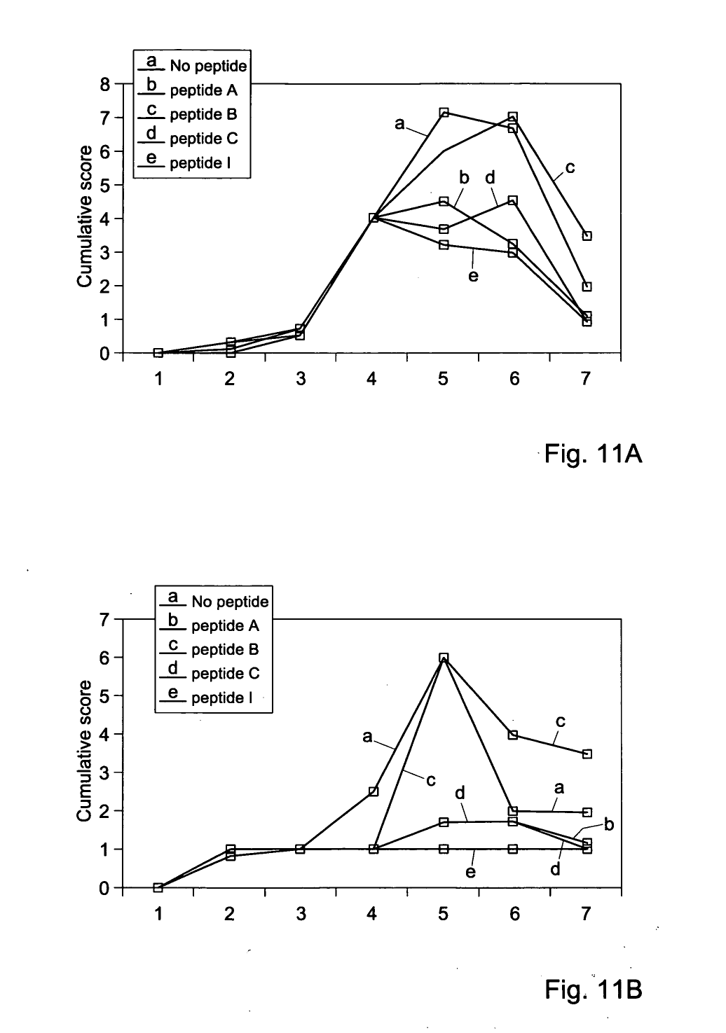

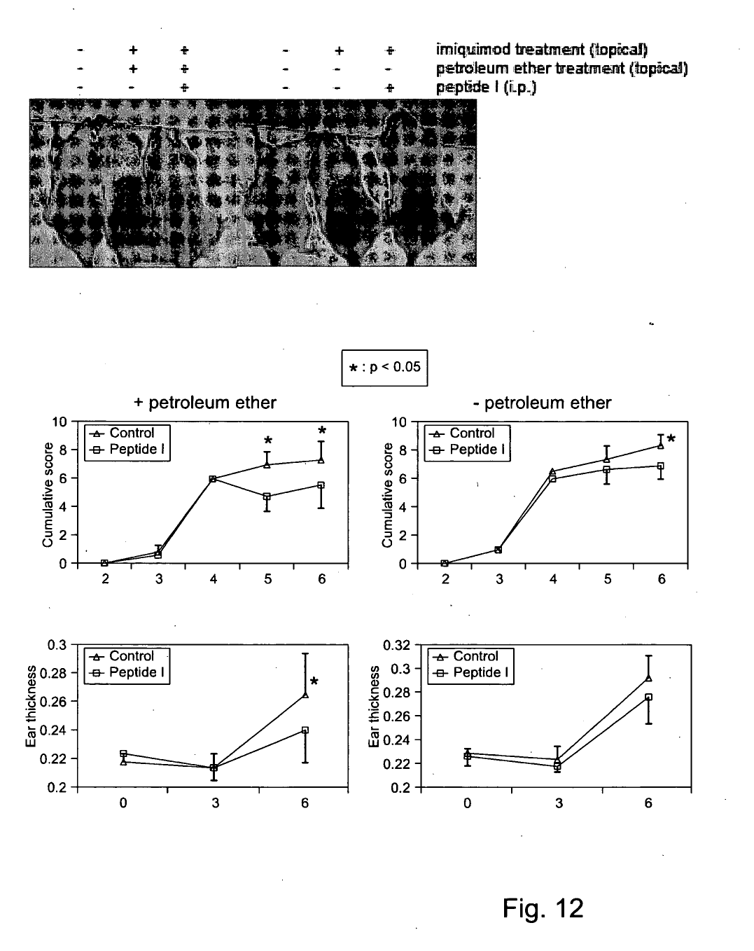

Peptides tested in this study were Peptide A (LAGV (SEQ ID NO:10)); Peptide B (AQGV (SEQ ID NO:2)); Peptide G (VLPALPQ (SEQ ID NO:13)) and Peptide I (LQGV). Peptides were given parenterally by intraperitoneal injection (i.p.). All peptides had beneficial activity on the imiquimod induced skin lesions, especially after the lesions had occurred (see FIGS. 11 and 12). Treatment with petroleum ether to remove fat and scales of the imiquimod induced lesions in one experiment improved the activity of peptide I (LQGV).

### Further Examples

The peptides LQGV (SEQ ID NO:1), MTR, MTRV (SEQ ID NO:20) (SEQ ID NO:20), AQGV (SEQ ID NO:2) (SEQ ID NO:2), LAGV (SEQ ID NO:10) (SEQ ID NO:10), AQG, LQG, VLPALPQ (SEQ ID NO:13), LAG, and VLPALP (SEQ ID NO:4) as mentioned herein were prepared by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the alpha-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP. Upon completion of the synthesis, the peptide resin was treated with a mixture of trifluoroacetic acid (TFA)/H₂O/triisopropylsilane (TIS) 95:2.5:2.5. After 30 minutes, TIS was added until decolorization. The solution was evaporated in vacuo and the peptide precipitated with diethyl ether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/minute; absorbance was detected from 190-370 nm. There were different gradient systems used. For example, for peptides LQG and LQGV (SEQ ID NO:1): ten minutes 100% A followed by linear gradient 0-10% B in 50 minutes. For example for peptides VLPALP (SEQ ID NO:4) and VLPALPQ (SEQ ID NO:13): five minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

### Abbreviations Used

LPS, lipopolysaccharide; IL, Interleukin; PGE prostaglandin E; EAE, experimental autoimmune encephalomyelitis; Th, T helper; ATCC American Type Culture Collection; IL-1ra, receptor antagonist; HLA human leukocyte antigen; TGF transforming growth factor; ELISA enzyme-linked immuno sorbent assay; COX cyclooxygenase; TNF, tumor necrosis factor; IFN interferon; MS Multiple sclerosis; CNS Central nervous system; NAWM Normal appearing white matter; MOG Myelin oligodendrocyte glycoprotein; ORO Oil red O;

TABLE I
Markers and antibodies.

| Molecule/marker | Function |
|---|---|
| IL-1ra | Anti-inflammatory, Endogenous IL-1 antagonist |
| IL-4 | Anti-inflammatory |
| PGES | Anti-inflammatory |
| TGF-beta | Anti-inflammatory |
| CCL18 | expressed by T/B cells, DC, macrophages, |
| | chemotactic to naïve T cells and iDC |
| HLA class II | Antigen presentation to CD4+ T cells |
| CD163 | Scavenger receptor for haptoglobin-hemoglobin |
| | complexes, anti-inflammatory actions |
| Mannose receptor | Lectin, recognition of micro-organisms |
| CD11b | Forms complement receptor 3 with CD18 |
| IL-1beta | Pro-inflammatory cytokine |
| TNF-alpha | Pro-inflammatory cytokine |
| IL-6 | Pro- and anti-inflammatory actions |
| IL-12 p40/p70 | Pro-inflammatory cytokine |
| MOG | Myelin oligodendrocyte glycoprotein |
| MAP-2 | Neuronal protein |

The invention is further explained with the aid of the following illustrative examples.

### Example 1

### Myelin-Laden Macrophages are Anti-Inflammatory Consistent with Foam Cells in Multiple Sclerosis

### Material and Methods

### Immunohistochemical Analysis of Postmortem MS Brain Tissue

Human autopsy brain tissue from 5 MS patients was provided by The Netherlands Brain Bank in Amsterdam. Immunohistochemistry was performed on frozen sections of MS brain tissue to detect expression of (anti-)inflammatory markers and CNS antigens (Table 1) as described previously (Hoefakker et al., 1995). In brief, 6 μm frozen sections were cut and thawed on to glass slides. Slides were kept overnight at room temperature in humidified atmosphere. After air-drying, slides were fixed in acetone containing 0.02% (v/v) H₂O₂. Slides were then air-dried for ten minutes, washed with PBS and incubated with optimally diluted primary antibody overnight at 4° C. in humidified atmosphere. Incubations with secondary rabbit anti-mouse-Ig-biotin (Dako) and tertiary horseradish peroxidase (HRP)-labeled avidin-biotin-complex (ABC/HRP: Dako) were performed for one hour at RT. HRP activity was revealed by incubation for ten minutes at RT with 3-amino-9-ethyl-carbazole (AEC: Sigma), leading to a bright red precipitate. After washing, sections were counterstained with hematoxylin, and embedded with glycerol-gelatin. Omission of primary antibody acted as control staining. Myelin degradation products were detected with oil-red O(ORO), which stains neutral lipids, as previously described (Chayen and Bitensky, 1991). The used antibodies were the anti-inflammatory markers IL-1ra (Biosource), IL-4 (U-Cytech), PGES (Cayman), TGF-beta (Santa Cruz), and CCL18 (R&D); for antigen recognition and presentation HLA class II (Dako), CD163, mannose receptor, CD11b (BD biosciences); as pro-inflammatory markers IL-1beta (gift from Dr. Boraschi), TNF-alpha (U-Cytech), IL-6 (Genzyme), IL-12p40/p70 (Pharmingen); for CNS proteins MOG, MAP-2 (Pierce).

### In Vitro Model for Myelin-Driven Foam Cell Formation

Myelin was isolated as described previously (Norton and Poduslo, 1973). In short, white matter derived from post-mortem brain tissue was homogenized in 0.32 M sucrose and subsequently layered on 0.85 M sucrose. After centrifugation at 75,000 g myelin was collected from the interface, washed in water and suspended in water for osmotic shock. Using this method, the purified myelin was shown to be free of any recognizable fragments of other subcellular elements. Previous studies have shown that purified myelin structurally resembled the whole multilamellar myelin structure surrounding as seen in tissue sections using electron microscopy (Autilio et al., 1964).

Peripheral blood mononuclear cells were isolated from heparinized blood from healthy donors using a Ficoll density gradient. Subsequently, monocytes were purified using Percoll density gradient resulting in >80% monocytes. Monocytes were cultured in suspension at a concentration of 1×10⁶ cells/ml in TEFLON® flasks (Nalgene) in RPMI with 5% human AB serum. After five to seven days monocyte-derived macrophages were recovered from the Teflon flasks and seeded in tissue culture plates. After 24 hours, non-adherent cells were removed and remaining cells were >95% macrophages as determined by macrophage-specific esterase staining. Foamy macrophages were generated in vitro by incubating macrophages with myelin for 24 hours to seven days (referred to as one day and seven day-old foamy macrophages). In most experiments 50 microg/ml myelin was used. Control macrophages were obtained from the same donor, and not fed with myelin.

### ELISA

To determine cytokine production in culture supernatants of foamy macrophages commercial capture ELISA was performed. TNF-alpha, IL-10 and IL-12p40 were measured in the collected culture supernatants. ELISA was performed according to the manufacturers' guidelines (Biosource). Briefly, polystyrene microtiter wells (Immuno Maxisorp) were coated overnight at 4° C. with monoclonal anti-cytokine capture antibodies. Wells were blocked for two hours at RT with PBS/0.5% BSA, followed by washing (0.9% NaCl/0.1% Tween20). Freshly thawed supernatants of the cell cultures and recombinant human cytokine-standards were incubated in duplicates for two hours at RT in the presence of a biotinylated second anti-cytokine detection antibody. After washing, wells were incubated with HRP-labeled poly-streptavidin (CLB) for 30 minutes at RT. HRP revelation was performed with 3,3′,5,5′-tetramethylbenzidine (TMB) peroxidase (KPL). Color development was stopped by adding equal volume of 1M H₂SO₄. Optical density was measured at 450 nm.

CCL18 levels were measured by sandwich ELISA assay using a commercially available CytoSet (Biosource), consisting of a capture-antibody, a biotinylated detection-antibody, recombinant CCL18 standard and streptavidin-HRP conjugate. Assay conditions were exactly as described by the manufacturer.

### Real-Time Quantitative PCR

To quantify mRNA expression by foamy macrophages total RNA was extracted from cell cultures using the GenElute Mammalian Total RNA kit (Sigma). RNA samples were treated with DNAse I (Invitrogen) to remove any contaminating DNA. Using 1 microg of the total RNA as template, copy DNA (cDNA) was prepared using the AMV Reverse Transcription System (Promega). To determine target gene mRNA expression, real-time quantitative reverse-transcription-PCR was performed using TaqMan technology (PE-Applied Biosystems) as described previously (van der Fits et al., 2003). Target gene expression levels were corrected for GAPDH mRNA levels. Sequences of the PCR primers (PE Biosystems), and fluorogenic probes (Eurogentec) are: forward primer 5′CCTTCCTCCTGTGCCTGATG3′ (SEQ ID NO:______), reverse primer 5′ACAATCTCATTTGAATCAGGAA3′ (SEQ ID NO:______), probe 5′TGCCCGACTCCCTTGGGTGTCA3′ (SEQ ID NO:______) for COX-2; forward primer 5′ACGGCGCTGTCATCGATT3′ (SEQ ID NO:______), reverse primer 5′GGCATTCTTCACCTGCTCCA3′ (SEQ ID NO:______), probe 5′CTTCCCTGTGAAAACAAGAGCAAGGCC3′ (SEQ ID NO:______) for IL-10; forward primer 5′GCCCAGGCAGTCAGATCATC3′ (SEQ ID NO:______), reverse primer 5′-GGGTTTGCTACAACATGGGCT3′ (SEQ ID NO:______), probe 5′CTCGAACCCCGAGTGACAAGCCTG3′ (SEQ ID NO:______) for TNF-α; forward primer 5′CACCGGAACGACATGGAGA3′ (SEQ ID NO:______), reverse primer 5′TCCAGGCGACAAAAGGGTTA3′ (SEQ ID NO:______), probe 5′TGGGCTTCGTCTACTCCTTTCTGGGTC 3′ (SEQ ID NO:______) for PGES; forward primer 5′GCCTGGCCTCCAGAAAGACC3′ (SEQ ID NO:______), reverse primer 5′ACCTGGTACATCTTCAAGTCTTCATAAAT3′ (SEQ ID NO:______), probe 5′CTTTTATGATGGCCCTGTGCCTTAGT3′ (SEQ ID NO:______) for IL-12p35; forward primer 5′GCCAGGAGTTGTGAGTTTCCA3′ (SEQ ID NO:______), reverse primer 5′-TGCAAGGCCCTTCATGATG3′ (SEQ ID NO:______), probe 5′TCTGACCACTTCTCTGCCTGCCCA3′ (SEQ ID NO:______) for CCL18. forward primer 5′-GTTCCCCATATCCAGTGTGG3′ (SEQ ID NO:______), reverse primer 5′-TCCTTTGCAAGCAGAACTGA3′ (SEQ ID NO:______), probe TGGCTGTG (Roche) for IL-23p19.

### Statistical Analysis

Statistical analysis was performed using the non-parametric Mann-Whitney analysis. P values<0.05 were considered significant.

Multiple sclerosis (MS) is a chronic inflammatory autoimmune disease of the central nervous system (CNS) and is characterized by the presence of demyelinated areas throughout the CNS (Sospedra and Martin, 2005). Various mechanisms leading to demyelination and axonal suffering have been implicated and the production of toxic inflammatory mediators by infiltrating and resident CNS macrophages is believed to play a pivotal role (Becher et al., 2000; Cannella and Raine, 2004; Lassmann, 2004; Matute and Perez-Cerda, 2005; Raine, 1994; Sospedra and Martin, 2005; Wingerchuk et al., 2001).

Different subsets of myeloid cells have distinct roles in the development of experimental autoimmune encephalomyelitis (EAE), an animal model for MS. These distinct and specialized roles of myeloid cells depend on their origin and, importantly, their location (Greter et al., 2005; Heppner et al., 2005; McMahon et al., 2005; Platten and Steinman, 2005). As such, perivascular cells appear to be optimally positioned for the modulation of infiltrating T cell activity whereas parenchymal myeloid cells may have a more prominent role in mechanisms involved in myelin breakdown and axonal suffering (Platten and Steinman, 2005).

The plasticity and functional polarization of macrophages have received renewed attention in light of novel key properties of different forms of macrophages. Two extremes of a continuum have been identified for macrophages, being M1, or classically activated macrophages, and M2, or alternatively activated macrophages (Gordon, 2003; Mantovani et al., 2004; Mantovani et al., 2002; Mosser, 2003). The M1 phenotype is typically induced in vitro by IFN-gamma, TNF-alpha or LPS, whereas the M2 phenotype can be induced by IL-10, IL-4 or by the lipid mediator PGE₂, which is a strong inhibitor of pro-inflammatory immune responses (Gratchev et al., 2001; Harris et al., 2002; Hinz et al., 2000; Ikegami et al., 2001; Kalinski et al., 1997). M1 macrophages are characterized by a high production of pro-inflammatory mediators and are involved in Th1 cell responses and killing of micro-organisms and tumor cells.

In contrast, M2 macrophages are associated with Th2 responses, scavenging of debris, promotion of tissue remodeling and repair and expression of anti-inflammatory molecules, including IL-1ra (IL-1 receptor antagonist) and CCL18 (Gordon, 2003; Mantovani et al., 2004). CCL18 in particular is a specific marker for human alternatively activated macrophages (Goerdt et al., 1999; Gordon, 2003; Kodelja et al., 1998; Mantovani et al., 2002) and is likely involved in immune suppression. Demyelinating MS lesions are characterized by the presence of foamy macrophages, a characteristic subset of myeloid cells, which acquire their distinctive morphology by ingestion and accumulation of vast amounts of myelin-derived lipids. Foamy macrophages originate from both resident microglia and infiltrating monocytes. 30-80% of foamy macrophages in demyelinating lesions are estimated to be blood-derived (Li et al., 1996). Besides their apparent role in scavenging myelin, it is still poorly understood if and how foamy macrophages may affect the local inflammatory process. Since MS lesions are self-limiting and do not expand indefinitely it is likely that local mechanisms restrict CNS inflammation and may also promote tissue repair. We hypothesized that foamy macrophages are anti-inflammatory M2-type macrophages and actively contribute to the resolution of brain inflammation and hence to tissue integrity and function. Our findings reveal an important and previously overlooked anti-inflammatory and modulatory role for foamy macrophages in MS lesions.

### Results of Example 1

### Foamy Macrophages Express Anti-Inflammatory Markers and Demonstrate a Unique Location-Dependent Phenotype

To determine the immune phenotype of lipid-laden foamy macrophages in MS lesions, we used antibodies against CNS proteins, various surface markers involved in antigen recognition and presentation, and pro- and anti-inflammatory markers characteristic for M1 and M2 macrophages (Goerdt et al., 1999; Gordon, 2003; Kodelja et al., 1998; Mantovani et al., 2002). Foamy macrophages were defined by their characteristic morphology, strong HLA-DR expression and presence of neutral lipids, which are detected by oil red 0 histochemistry (ORO). To determine whether foamy macrophages display phenotypic and functional specialization dependent on micro-location, we analyzed the phenotype of these cells in different micro-locations. We distinguished between foamy macrophages within the lesion, in perivascular spaces within the lesion and in the outer or inner rim. The distinction between the outer and inner rim was based on the presence of neutral lipids, MOG and on the size of the foamy macrophages. Outer rim foamy macrophages were smaller in size and contained more MOG, but less neutral lipids than inner rim foamy macrophages.

IL-6, a cytokine with pro-as well as anti-inflammatory properties as well as the anti-inflammatory M2 marker IL-1ra and prostaglandin E₂ synthase (PGES) were differentially expressed in the distinct areas of an MS-lesion. Whereas IL-6 and IL-1ra were detected mostly in perivascular and lesional foamy macrophages, PGES was mostly expressed in the outer, and to a lesser extent in the inner rim. Importantly, expression patterns between cells varied even when cells were in close proximity. Mannose receptor, which is characteristic for M2 macrophages (Gordon, 2003; Mantovani et al., 2004; Mantovani et al., 2002; Mosser, 2003), was highly expressed on foamy macrophages in perivascular spaces but was mostly absent on parenchymal foamy macrophages. Occasionally, a weakly positive cell was observed which was always in the vicinity of a blood vessel. TGF-beta expression showed the reverse expression pattern with more pronounced expression by parenchymal foamy macrophages compared to perivascular foamy macrophages.

As hypothesized, the relative levels of expression were related to specific micro-locations within the lesion. Foamy macrophages in the lesion rim contained MOG, and immunoreactivity showed a decreasing trend towards the center of the lesion, possibly reflecting time-dependent myelin degradation. In contrast, intracellular neuronal antigen MAP-2 immunoreactivity increased towards the center of the lesion, implicating that neuronal damage occurs mostly in the lesion center. Only foamy macrophages within perivascular spaces expressed the surface markers CD11b, CD163 and mannose receptor. The anti-inflammatory molecules IL-1ra, CCL18, IL-10, TGF-beta and IL-4 were all strongly expressed by foamy macrophages, and expression was highest in the center of the lesion. Interestingly, IL-10 expression was absent on foamy macrophages in perivascular spaces. The pro-inflammatory cytokines TNF-alpha, IL-1beta, IL-12p40/70 were not expressed by foamy macrophages in any of the micro-locations, whereas cells associated with vessels in normal appearing white matter (NAWM) did express these pro-inflammatory cytokines. Phenotypic heterogeneity was not observed among non-foamy macrophages that were present in low numbers in perivascular spaces in NAWM.

Thus, we demonstrate that foamy macrophages in the brain have clear anti-inflammatory characteristics, resemble M2 macrophages, and have a unique phenotype depending on the micro-location.

Myelin Induces a Foamy Morphology in Macrophages Resembling that of Foamy Macrophages In Situ

Next, we set out to determine whether ingestion of myelin in vitro results in an anti-inflammatory function of foamy macrophages as observed in situ. Therefore, we first developed a fully human in vitro model of foamy macrophages. In short, human monocyte-derived macrophages are cultured in the absence or presence of human brain-derived myelin for 24 hours. Whereas cells cultured in the absence of myelin did not appear foamy (at magnification 32×), those cultured with myelin acquire a characteristic foamy morphology as observed by light microscopy. Human primary macrophages obtained from healthy donors were fed with 50 microg/ml human myelin and changes in the morphology were monitored by light microscopy and by ORO staining to detect intracellular neutral lipids. Although small individual changes in kinetics between individual donors were observed, macrophages acquired a foamy morphology between 24 and 48 hours and contained a markedly increased number and size of lipid droplets in comparison to control macrophages (i.e., not fed with myelin) as demonstrated by ORO staining. The typical foamy morphology of macrophages could still be observed one week upon the initial addition of myelin. Macrophage viability was not affected by myelin ingestion when a dose range of 1-100 microg/ml as was used, as was demonstrated by trypan blue staining.

### Foamy Macrophages do not Mount Pro-Inflammatory Responses to Prototypical Inflammatory Stimuli and Produce Anti-Inflammatory Mediators

To assess the effect of myelin ingestion on macrophage function, cytokine levels were determined in supernatants of myelin-laden macrophages before and after LPS stimulation. Since variation in myelin lipid composition between MS and normal brain has been reported (Woelk and Borri, 1973), myelin was isolated from white matter of three control brains and three MS brains to investigate possible functional differences. Macrophages were incubated with the distinct myelin preparations for 24 hours and IL-10 and IL-12p40 levels were determined in the supernatants by ELISA. None of the myelin preparations induced IL-12p40 and only the highest dose of one MS brain-derived myelin was associated with a transient IL-10 induction. All myelin preparations inhibited LPS-induced IL-12p40 and IL-10 induction in a dose-dependent fashion. No significant differences were observed in cytokine production between foamy macrophages generated using the different myelin preparations. For subsequent experiments 50 microg/ml myelin was used.

Next, the effect of myelin ingestion on LPS-induced mRNA levels of different pro- and anti-inflammatory mediators was determined. Macrophages were incubated with myelin for 24 hours and subsequently stimulated with LPS for an additional two hours, after which RNA was isolated and real time RT-PCR was performed for IL-12p35, TNF-alpha, IL-10, COX-2, PGES and CCL18. LPS-induced IL-12p35 and TNF-alpha expression by foamy macrophages was completely inhibited. IL-10 was slightly but not significantly induced by LPS in control macrophages as well as foamy macrophages. COX-2 was increased after LPS stimulation in control macrophages but this induction was not significantly inhibited in foamy macrophages. Foamy macrophages showed between 15-50 and 8-12-fold induction of CCL18 and PGES compared to control macrophages. Thus, myelin ingestion resulted in a differential modulation of LPS responses. LPS-induced IL-12p40 and TNF-alpha expression was strongly and significantly inhibited, IL-10 and COX-2 expression remained unaffected and the expression of anti-inflammatory CCL18 and PGES significantly increased.

To determine whether myelin ingestion results in long-term modulation of macrophage function, macrophages were incubated with myelin for the indicated time periods and real time RT-PCR was performed for IL-12p35, IL-10, PGES and CCL18. IL-10 mRNA was not detectable at any time point. After myelin uptake IL-12p35 expression was decreased, albeit not significantly, over time in comparison to control macrophages. In contrast to IL-12p35 both PGES and CCL18 were induced by myelin. Seven day-old foamy macrophages expressed ten- and 90-fold more PGES and CCL18 than control macrophages. IL-12p40, IL-10, and CCL18 levels were subsequently determined in supernatants of these foamy macrophages. CCL18 is constitutively produced by macrophages and production by foamy macrophages is increased at day 7 after myelin ingestion, paralleling the increased CCL18 mRNA expression by foamy macrophages. IL-12p40 and IL-10 were not detectable.

Subsequently we determined whether the aberrant LPS response persisted over time. Seven days after initial myelin ingestion foamy macrophages were stimulated with 1 ng/ml LPS for 24 hours and cytokine levels in the supernatant were determined by ELISA. LPS-induced IL-12p40 and IL-10 production by these foamy macrophages was abolished completely whereas CCL18 was significantly increased. In addition, responses to other prototypical pro-inflammatory stimuli such as peptidoglycan and zymosan were also completely abolished.

The relapsing-remitting nature of MS strongly suggests the presence of potent counter-regulatory mechanisms that keep the disease in check. One such mechanism may be the active control of inflammation in the CNS itself thus preventing infinite expansion of the demyelinating lesion. Inflammation and demyelination are responsible for at least short-term neurological symptoms. Inflammation probably contributes to axonal loss as neurons are more vulnerable to environmental insults when the protective myelin sheaths are destroyed and the axons exposed (Grigoriadis et al., 2004; Kuhlmann et al., 2002). It is therefore imperative that in the developing lesions the production of toxic molecules is halted and that inflammation is limited allowing for tissue repair (Sospedra and Martin, 2005).

Myelin-laden foamy macrophages are abundantly present in demyelinating lesions and although it is generally assumed that these cells contribute to inflammation, evidence for this is scarce (van der Laan et al., 1996). This lack of data on foamy macrophage function in MS is in sharp contrast with the increasing attention for foam cells in atherosclerosis (Greaves and Gordon, 2005) reporting potent immune-regulatory functions by lipids and lipid-induced molecules (Harris et al., 2002; Joseph et al., 2004; Joseph et al., 2003; Lawrence et al., 2002; Pettus et al., 2002). Lipid-laden cells are anti-inflammatory (Lawrence et al., 2002) and it was shown that low-density lipoprotein (LDL) uptake by macrophages inhibits TNF-induced TNF expression and induces IL-10 (Ares et al., 2002; Lo et al., 1999; Varadhachary et al., 2001). Foamy macrophages in the rim of active demyelinating lesions have been shown to contain plasma LDL (Newcombe et al., 1994).

Here, we establish that foamy macrophages in active MS lesions have consistent immunosuppressive function, while displaying a unique surface phenotype dependent on the micro-location. In addition, we demonstrate that ingestion of human myelin alters human macrophage function in vitro by inducing anti-inflammatory molecules and by inhibiting responses to pro-inflammatory stimuli. The results presented here reveal a new regulatory pathway in MS.

We show here that the observed functional phenotype of foamy macrophages in MS lesions results from the accumulation of lipids derived from myelin and phagocytosed apoptotic cell membranes, in concert with local microenvironmental cues, such as differences in extracellular matrix content in the perivascular infiltrate versus the lesion in the brain parenchyma. Foamy macrophages demonstrate a phenotype resembling that of anti-inflammatory M2 macrophages, are likely to contribute to resolution of inflammation, and may therefore be responsible for inhibiting further lesion development and promoting lesion repair. In addition, they may also function as a first line of defense against infiltrating inflammatory myeloid cells. Future studies are required to elucidate which lipid components are able to regulate macrophage function and which mechanisms are involved. Understanding the mechanisms behind naturally occurring counter-regulatory processes allows for definition of new cellular targets for therapeutic drug design for the treatment of MS and even has broader applications for other foam cell-associated diseases including atherosclerosis and lung-conditions.

### Example 2

### Determining Whether Compounds Modulate Responses by Macrophages and Foam Cells Experimental Design

Human monocyte-derived macrophages were cultured in medium (=macrophages) or in the presence of human brain-derived myelin for 48 hours (=foam cells).

Macrophages and foam cells were cultured in the presence of 10 microg/ml compounds LAGV (SEQ ID NO:10) (SEQ ID NO:10), AQGV (SEQ ID NO:2) (SEQ ID NO:2), LAG, AQG, MTR, MTRV (SEQ ID NO:20) (SEQ ID NO:20), VLPALPQ (SEQ ID NO:13), VLPALP (SEQ ID NO:4), LQGV (SEQ ID NO:1), LQG (see for example PCT International Publication No. WO 03/029292 A2 (published Apr. 10, 2003), PCT International Publication No. WO 01/72831 A2 (published Oct. 4, 2001), PCT/EP2004/003747, and U.S. Patent Application Publications 20020064501 A1 (published May 30, 2002), 20030119720 A1 (published Jun. 26, 2003), 20030113733 A1 (published Jun. 19, 2003), US 2003/0220259 A1 (published Nov. 27%, 2003) and 20030166556 A1 (published Sep. 4, 2003), the contents of all of which are incorporated by this reference) for three hours.

10 ng/ml LPS was added to the cultures for an additional 16 hours.

Supernatants were collected and ELISA performed for TNF-alpha, IL-12p40, and IL-10.

### Results:

Protein levels are depicted in Table 2.

LPS induced TNF-alpha, IL-12p40 and IL-10 in macrophages as expected, confirming the experimental system performed as usual.

Foam cells demonstrated decreased LPS responses for IL-10 and IL-12p40 as expected. LPS-induced TNF-alpha production by foam cells was not affected as has been observed before.

Effects of compounds on LPS responses are shown in Table 1.

The compounds did not affect macrophage or foam cell morphology or viability as judged by microscopic examination.

### Example 3

### Determining Whether Compounds Affect Cytokine Production by Human Macrophages and Foam Cells Experimental Design

Human monocyte-derived macrophages from a healthy blood bank donor were cultured in medium (=macrophages) or in the presence of human brain-derived myelin for 48 hours (=foam cells).

Macrophages and foam cells were cultured in duplicate in the presence of 10 microg/ml of compounds LAGV (SEQ ID NO:10) (SEQ ID NO:10), AQGV (SEQ ID NO:2) (SEQ ID NO:2), LAG, AQG, MTR, MTRV (SEQ ID NO:20) (SEQ ID NO:20), VLPALPQ (SEQ ID NO:13), VLPALP (SEQ ID NO:4), LQGV (SEQ ID NO:1), LQG for two or eight hours, or cultured in macrophage medium with vehicle.

Cells were lysed and real time RT-PCR (TaqMan technology) was performed on all samples for GAPDH (housekeeping gene), TNF-alpha (pro-inflammatory), IL-12p35 (pro-inflammatory), IL-10 (anti-inflammatory), CCL18 (chemokine), COX-2 (prostaglandin pathway).

### Results:

Effects of compounds on mRNA expression levels are depicted in Tables 2, 3 and 4.

The compounds did not affect macrophage or foam cell morphology or viability as judged by microscopic examination.

cDNA quality of two samples was not sufficient for reliable semi-quantification. Values of these samples (#6, peptides two hours on macrophages; # 10, peptides eight hours on foam cells) have been omitted.

### Example 4

### Determining Whether Foam Cells Differentially Express Chemokines Compared to Control Macrophages Experimental Design

Human monocyte-derived macrophages from a healthy blood bank donor were cultured in medium (=macrophages) or in the presence of human brain-derived myelin for 48 hours (=foam cells)

10 microg/ml of the compounds LAGV (SEQ ID NO:10) (SEQ ID NO:10), AQGV (SEQ ID NO:2) (SEQ ID NO:2) or LQGV (SEQ ID NO:1) is added to macrophages or foam cells for six hours, or macrophages or foam cells are cultured in macrophage medium with vehicle.

Cells were lysed, RNA isolated and Affymetrix microarray (U133+2 chip with 53.675 transcripts) was used according to the manufacturer's instructions to determine relative mRNA levels

### Results:

Effect of myelin ingestion and additional effect of the compounds LAGV (SEQ ID NO:10) (SEQ ID NO:10), AQGV (SEQ ID NO:2) (SEQ ID NO:2) or LQGV (SEQ ID NO:1) on eight different selected chemokines is depicted in Table 6.

Compounds did not affect the chemokine expression by control macrophages.

### Example 5

The efficiency of ten different peptides was testes in four assays (as described in Examples 1 and 2). Peptides were ranked in order of efficiency, with one being the effect considered to be most beneficial for MS patients (i.e., low TNF, high IL-10, high CCL18) and ten being the most detrimental (i.e., high TNF, low IL-10, low CCL18). For each peptide, the mean ranking was calculated. The peptide with the highest overall ranking (i.e., the lowest number) is the most potent peptide with regards to the induction of effects which can be considered beneficial for MS patients. Results are shown in Table 7.

TABLE 2

LPS-induced cytokine responses by compounds
tested in human macrophages and in foam
cells

| | | | | |
|---|---|---|---|---|
| | Mean | Mean | sd | sd |
| | macro- | foam | macro- | foam |
| compound | phages | cells | phages | cells |

TNF-alpha (pg/ml)

| | | | | | |
|---|---|---|---|---|---|
| None | | 21664 | 29366 | 2532 | 2733 |
| LAGV | (SEQ ID NO:10) | 8336 | 16464 | 322 | 2331 |
| AQGV | (SEQ ID NO:2) | 9075 | 15895 | 1688 | 723 |
| LAG | 13281 | 15895 | 2009 | 5225 | |
| AQGV | (SEQ ID NO:2) | 14475 | 14531 | 482 | 563 |
| MTR | 13054 | 15839 | 2170 | 5626 | |
| MTRV | (SEQ ID NO:20) | 14816 | 19249 | 3697 | 804 |
| VLPALPQ | (SEQ ID NO:13) | 13167 | 20101 | 402 | 563 |
| VLPALP | (SEQ ID NO:4) | 10723 | 20670 | 4823 | 6189 |
| LQGV | (SEQ ID NO:1) | 5381 | 13565 | 1125 | 482 |
| LQG | 4812 | 16691 | 643 | 884 | |

IL-10 (pg/ml)

| | | | | | |
|---|---|---|---|---|---|
| None | | 4140 | 485 | 118 | 29 |
| LAGV | (SEQ ID NO:10) | 2203 | 222 | 358 | 29 |
| AQGV | (SEQ ID NO:2) | 2977 | 186 | 105 | 0 |
| LAG | 2793 | 191 | 65 | 2 | |
| AQGV | (SEQ ID NO:2) | 2078 | 179 | 110 | 0 |
| MTR | 2399 | 206 | 18 | 2 | |
| MTRV | (SEQ ID NO:20) | 3105 | 235 | 251 | 11 |
| VLPALPQ | (SEQ ID NO:13) | 3004 | 229 | 107 | 16 |
| VLPALP | (SEQ ID NO:4) | 2654 | 153 | 11 | 20 |
| LQGV | (SEQ ID NO:1) | 3654 | 572 | 96 | 13 |
| LQG | 4209 | 601 | 172 | 22 | |

IL-12p40 (pg/ml)

| | | | |
|---|---|---|---|
| None | | 2258 | 1320 |
| LAGV | (SEQ ID NO:10) | 2038 | 1100 |
| AQGV | (SEQ ID NO:2) | 1527 | 1093 |
| LAG | 1799 | 899 | |
| AQGV | (SEQ ID NO:2) | 1942 | 997 |
| MTR | 1910 | 912 | |
| MTRV | (SEQ ID NO:20) | 2325 | 804 |
| VLPALPQ | (SEQ ID NO:13) | 1901 | 1218 |
| VLPALP | (SEQ ID NO:4) | 2426 | 1284 |
| LQGV | (SEQ ID NO:1) | 3364 | 1274 |
| LQG | 1859 | 1742 | |

TABLE 3
Taqman results

| | | | | | |
|---|---|---|---|---|---|
| CCL18 | | | CCL18 | | |
| peptide | | | peptide | | |
| treat- | | | treat- | | |
| ment | | | ment | | |
| 2 hrs | mean | s.d. | 8 hrs | mean | s.d. |

| | | | | | | |
|---|---|---|---|---|---|---|
| macro- | None | 0.99 | 0.45 | None | 1.14 | 0.29 |
| phages | LAGV | 1.00 | 0.13 | LAGV | 1.28 | 0.50 |
| | (SEQ ID NO:10) | | | (SEQ ID NO:10) | | |
| | AQGV | 1.11 | 0.39 | AQGV | 2.42 | 0.16 |
| | (SEQ ID NO:2) | | | (SEQ ID NO:2) | | |
| | LAG | 1.70 | 0.52 | LAG | 1.29 | 0.14 |
| | AQG | 1.00 | 0.43 | AQG | 1.91 | 0.25 |
| | MTR | ND | | MTR | 1.46 | 0.20 |
| | MTRV | 1.74 | 0.14 | MTRV | 1.13 | 0.39 |
| | (SEQ ID NO:20) | | | (SEQ ID NO:20) | | |
| | VLPALPQ | 2.50 | 0.32 | VLPALPQ | 0.98 | 0.48 |
| | (SEQ ID NO:13) | | | (SEQ ID NO:13) | | |
| | VLPALP | 1.31 | 0.20 | VLPALP | 2.20 | 0.48 |
| | (SEQ ID NO:4) | | | (SEQ ID NO:4) | | |
| | LQGV | 2.41 | 1.04 | LQGV | 2.29 | 0.53 |
| | (SEQ ID NO:1) | | | (SEQ ID NO:1) | | |
| | LQG | 1.33 | 0.31 | LQG | 1.91 | 0.10 |
| foam | None | 278.87 | 99.48 | None | 21.07 | 3.08 |
| cells | LAGV | 886.13 | 353.99 | LAGV | 81.82 | 9.06 |
| | (SEQ ID NO:10) | | | (SEQ ID NO:10) | | |
| | AQGV | 600.00 | 211.58 | AQGV | 52.94 | 11.11 |
| | (SEQ ID NO:2) | | | (SEQ ID NO:2) | | |
| | LAG | 219.51 | 33.08 | LAG | 92.32 | 31.57 |
| | AQG | 355.94 | 81.34 | AQG | 118.82 | 29.83 |
| | MTR | 262.81 | 65.57 | MTR | 124.05 | 24.11 |
| | MTRV | 277.78 | 94.19 | MTRV | 42.65 | 0.00 |
| | (SEQ ID NO:20) | | | (SEQ ID NO:20) | | |
| | VLPALPQ | 205.43 | 46.94 | VLPALPQ | 49.25 | 3.90 |
| | (SEQ ID NO:13) | | | (SEQ ID NO:13) | | |
| | VLPALP | 278.99 | 2.01 | VLPALP | 55.00 | 16.88 |
| | (SEQ ID NO:4) | | | (SEQ ID NO:4) | | |
| | LQGV | 488.87 | 38.70 | LQGV | ND | |
| | (SEQ ID NO:1) | | | (SEQ ID NO:1) | | |
| | LQG | 153.76 | 8.86 | LQG | 32.86 | 8.42 |

TABLE 4
Taqman results

| | | | | | |
|---|---|---|---|---|---|
| COX-2 | | | COX-2 | | |
| Peptide | | | peptide | | |
| treat- | | | treat- | | |
| ment | | | ment | | |
| 2 hrs | mean | s.d. | 8 hrs | mean | s.d. |

| | | | | | | |
|---|---|---|---|---|---|---|
| macro- | None | 0.591211 | 0.226438 | None | 0.94 | 0.32 |
| phages | LAGV | 1.28552 | 0.590098 | LAGV | 1.47 | 1.33 |
| | (SEQ ID NO:10) | | | (SEQ ID NO:10) | | |
| | AQGV | 1.009161 | 0.171062 | AQGV | 0.71 | 0.03 |
| | (SEQ ID NO:2) | | | (SEQ ID NO:2) | | |
| | LAG | 1.047836 | 0.344225 | LAG | 1.42 | 0.10 |
| | AQG | 1.28199 | 0.434554 | AQG | 1.58 | |
| | MTR | ND | | MTR | 2.40 | 1.05 |
| | MTRV | 1.321293 | 0.966936 | MTRV | 0.71 | 0.39 |
| | (SEQ ID NO:20) | | | (SEQ ID NO:20) | | |
| | VLPALPQ | 1.01643 | 0.301701 | VLPALPQ | 0.50 | 0.15 |
| | (SEQ ID NO:13) | | | (SEQ ID NO:13) | | |
| | VLPALP | 1.03924 | 0.186638 | VLPALP | 0.65 | 0.10 |
| | (SEQ ID NO:4) | | | (SEQ ID NO:4) | | |
| | LQGV | 0.577613 | 0.104948 | LQGV | 0.67 | 0.12 |
| | (SEQ ID NO:1) | | | (SEQ ID NO:1) | | |
| | LQG | 0.673839 | 0.100383 | LQG | 2.27 | 2.00 |
| foam | None | 0.89199 | 0.159893 | None | 0.79 | 0.09 |
| cells | LAGV | 0.912541 | 0 | LAGV | 2.19 | 0.46 |
| | (SEQ ID NO:10) | | | (SEQ ID NO:10) | | |
| | AQGV | 0.763449 | 0.203646 | AQGV | 2.31 | 0.28 |
| | (SEQ ID NO:2) | | | (SEQ ID NO:2) | | |
| | LAG | 0.722072 | 0.2582 | LAG | 1.66 | 0.50 |
| | AQG | 1.081277 | 0.053871 | AQG | 1.77 | 0.30 |
| | MTR | 0.73216 | 0.14501 | MTR | 2.48 | 0.01 |
| | MTRV | 1.445971 | 0 | MTRV | 2.15 | 0.53 |
| | (SEQ ID NO:20) | | | (SEQ ID NO:20) | | |
| | VLPALPQ | 0.690174 | 0 | VLPALPQ | 1.15 | 0.07 |
| | (SEQ ID NO:13) | | | (SEQ ID NO:13) | | |
| | VLPALP | 1.013483 | 0.109235 | VLPALP | 1.44 | 1.46 |
| | (SEQ ID NO:4) | | | (SEQ ID NO:4) | | |
| | LQGV | 0.805138 | 0.046792 | LQGV | ND | |
| | (SEQ ID NO:1) | | | (SEQ ID NO:1) | | |
| | LQG | 0.556831 | 0.159653 | LQG | 0.88 | 0.63 |

TABLE 5
Taqman results

| | | | | | |
|---|---|---|---|---|---|
| IL-10 | | | IL-10 | | |
| peptide | | | peptide | | |
| treat- | | | treat- | | |
| ment | | | ment | | |
| 2 hrs | mean | s.d. | 8 hrs | mean | s.d. |

| | | | | | | |
|---|---|---|---|---|---|---|
| macro- | None | 0.838719 | 0.084748 | None | 0.96 | 0.23 |
| phages | LAGV | 0.93201 | 0.073727 | LAGV | 1.25 | 0.31 |
| | (SEQ ID NO:10) | | | (SEQ ID NO:10) | | |
| | AQGV | 1.078281 | 0.162801 | AQGV | 1.97 | 0.17 |
| | (SEQ ID NO:2) | | | (SEQ ID NO:2) | | |
| | LAG | 0.949998 | 0.155105 | LAG | 1.14 | 0.10 |
| | AQG | 0.794645 | 0.120295 | AQG | 1.28 | 0.23 |
| | MTR | ND | | MTR | 1.56 | 0.77 |
| | MTRV | 0.799543 | 0.059873 | MTRV | 0.83 | 0.20 |
| | (SEQ ID NO:20) | | | (SEQ ID NO:20) | | |
| | VLPALPQ | 0.95685 | 0.180639 | VLPALPQ | 1.29 | 0.46 |
| | (SEQ ID NO:13) | | | (SEQ ID NO:13) | | |
| | VLPALP | 0.79994 | 0.060407 | VLPALP | 1.39 | 0.25 |
| | (SEQ ID NO:4) | | | (SEQ ID NO:4) | | |
| | LQGV | 0.623505 | 0.04854 | LQGV | 0.81 | 0.20 |
| | (SEQ ID NO:1) | | | (SEQ ID NO:1) | | |
| | LQG | 0.604754 | | LQG | 1.91 | 0.80 |
| foam | None | 0.680602 | 0.118096 | None | 0.471046 | 0.074507 |
| cells | LAGV | 0.628717 | 0.031388 | LAGV | 1.34 | 0.19 |
| | (SEQ ID NO:10) | | | (SEQ ID NO:10) | | |
| | AQGV | 0.788278 | 0.120264 | AQGV | 1.34 | 0.42 |
| | (SEQ ID NO:2) | | | (SEQ ID NO:2) | | |
| | LAG | 0.564875 | 0.022564 | LAG | 0.91 | 0.10 |
| | AQG | 0.724893 | 0.06027 | AQG | 1.16 | 0.16 |
| | MTR | 0.801344 | 0.047998 | MTR | 0.99 | 0.05 |
| | MTRV | 0.936911 | 0.309266 | MTRV | 1.07 | 0.12 |
| | (SEQ ID NO:20) | | | (SEQ ID NO:20) | | |
| | VLPALPQ | 0.56192 | 0.028053 | VLPALPQ | 1.01 | 0.17 |
| | (SEQ ID NO:13) | | | (SEQ ID NO:13) | | |
| | VLPALP | 0.745603 | 0.103929 | VLPALP | 0.83 | 0.05 |
| | (SEQ ID NO:4) | | | (SEQ ID NO:4) | | |
| | LQGV | 0.682842 | 0.090676 | LQGV | ND | |
| | (SEQ ID NO:1) | | | (SEQ ID NO:1) | | |
| | LQG | 0.518613 | 0.032787 | LQG | 1.01 | 0.04 |

TABLE 6
Fold differences of selected chemokines as determined by Affymetrix microarray

| | | Additional effect | | Additional effect |
|---|---|---|---|---|
| | | of LAGV (SEQ | Additional effect | of LQGV (SEQ |
| | Effect of myelin | ID NO: 10) | of AQGV | ID NO: 1) |
| Chemokine | ingestion | (SEQ ID NO: 10) | (SEQ ID NO: 2) | (SEQ ID NO: 1) |
| CCL2 | −1.6 | 3.27 | 5.3 | 3.1 |
| CCL3 | 2.5 | 1.1 | 1.1 | 1.0 |
| CCL4 | 3.3 | 1.3 | 1.4 | 1.1 |
| CCL5 | 4.3 | 1.4 | 2.1 | 1.7 |
| CCL7 | 1.6 | 1.6 | 2.5 | 1.9 |
| CXCL3 | 1.0 | 1.8 | 2.3 | 1.9 |
| CXCL8 | 5.1 | 1.5 | 2.0 | 1.8 |
| CCL18 | 3 | 2.8 | 4.5 | 3.3 |

TABLE 7
Ranking of peptides as to suitability for treatment of MS

| | mean | ranking | ranking | ranking | ranking |
|---|---|---|---|---|---|
| | ranking | a | b | c | d |
| LQGV (SEQ ID | 2.25 | 1 | 2 | 3 | 3 |
| NO: 1) | | | | | |
| MTR | 4.25 | 3 | 6 | 1 | 7 |
| MTRV (SEQ ID NO: 20) | 5.25 | 8 | 3 | 4 | 6 |
| AQGV (SEQ ID | 5.5 | 4 | 8 | 8 | 2 |
| NO: 2) | | | | | |
| LAGV (SEQ ID | 5.5 | 6 | 5 | 10 | 1 |
| NO: 10) | | | | | |
| AQG | 5.75 | 2 | 9 | 9 | 3 |
| LQG | 6 | 7 | 1 | 6 | 10 |
| VLPALPQ (SEQ ID | 6 | 9 | 4 | 2 | 9 |
| NO: 13) | | | | | |
| LAG | 6.25 | 5 | 7 | 5 | 8 |
| VLPALP (SEQ ID | 8 | 10 | 10 | 7 | 5 |
| NO: 4) | | | | | |

Ranking parameters
A lowest TNF foamy macrophages
B highest IL-10 foamy macrophages
C highest CCL18 macrophages
D highest CCL18 foamy macrophages

### REFERENCES

1. Vincent J. L., J. Carlet, and S. M. Opal, eds., The sepsis text, 2002.
2. Cohen J. The immunopathogenesis of sepsis. Nature 2002, 420:885-891.
3. Osborn T. M., J. K. Tracy, J. R. Dunne, M. Pasquale, and L. M. Napolitano. Epidemiology of sepsis in patients with traumatic injury. Crit. Care Med. 2004, 32(11):2234-40.
4. DS, R. Lefering, and C. E. Wade. Impact of hemorrhage on trauma outcome: an overview of epidemiology, clinical presentations, and therapeutic considerations. J. Trauma, 2006, 60(6):S3-11.
5. Muylle L. The role of cytokines in blood transfusion reactions. Blood Rev. 1995, 9:77-83.
6. Matsutani T., S. C. Kang, M. Miyashita, K. Sasajima, M. A. Choudhry, K. I. Bland, et al. Liver cytokine production and ICAM-1 expression following bone fracture, tissue trauma, and hemorrhage in middle-aged mice. Am. J. Physiol. Gastrointest. Liver Physiol. 2007, 292:268-74.
7. Rothoerl R. D., K. M. Schebesch, M. Kubitza, C. Woertgen, A. Brawanski, and A. L. Pina. ICAM-1 and VCAM-1 expression following aneurismal subarachnoid hemorrhage and their possible role in the pathophysiology of subsequent ischemic deficits. Cerebrovasc. Dis. 2006, 22:143-9.
8. Sun L. L., P. Ruff, B. Austin, S. Deb, B. Martin, D. Burris, et al. Early up-regulation of intercellular adhesion molecule-1 and vascular cell adhesion molecule-1 expression in rats with hemorrhagic shock and resuscitation. Shock 1999, 11:416-22.
9. Ferguson K. L., P. Taheri, J. Rodriguez, V. Tonapi, A. Cardellio, and R. Dechert. Tumor necrosis factor activity increases in the early response to trauma. Acad. Emerg. Med. 1997, 4(11):1035-40.
10. Mainous M. R., W. Ertel, I. H. Chaudry, and E. A. Deitch. The gut: a cytokine-generating organ in systemic inflammation? Shock 1995, 4:193-9.
11. Yang R., X. Han, T. Uchiyama, S. K. Watkins, A. Yaguchi, R. L. Delude, and M. P. Fink. IL-6 is essential for development of gut barrier dysfunction after hemorrhagic shock and resuscitation in mice. Am. J. Physiol. Gastrointest. Liver Physiol. 2003, 285(3):G621-9.
12. Taniguchi T., Y. Koido, J. Aiboshi, T. Yamashita, S. Suzaki, and A. Kurokawa. Change in the ratio of interleukin-6 to interleukin-10 predicts a poor outcome in patients with systemic inflammatory response syndrome. Crit. Care Med. 1999, 27:1262-1264.
13. Snyder E. L. The role of cytokines and adhesive molecules in febrile non-hemolytic transfusion reactions. Immunol. Invest. 1995, 24:333-9.
14. Winkelstein A., and J. E. Kiss. Immunohematologic disorders. Jama. 1997, 278:1982-92.
15. Davenport R. Cytokines and erythrocyte incompatibility. Curr. Opin. Hematol. 1994, 1:452-6.
16. Davenport R. D. The role of cytokines in hemolytic transfusion reactions. Immunol. Invest. 1995, 24:319-31.
17. Zhu X. L., R. Zellweger, X. H. Zhu, A. Ayala, and I. H. Chaudry. Cytokine gene expression in splenic macrophages and Kupffer cells following hemorrhage. Cytokine 1995, 7(1):8-14.
18. Fong Y., K. J. Tracey, L. L. Moldawer, D. G. Hesse, K. B. Manogue, J. S. Kenney, A. T. Lee, G. C. Kuo, A. C. Allison, S. F. Lowry, and C. A. Cermani. Antibodies to cachectin/tumor necrosis factor reduce interleukin 1 beta and interleukin 6 appearance during lethal bacteremia. J. Exp. Med. 1989, 1; 170(5):1627-33.
19. Robinson M. K., J. D. Rounds, R. W. Hong, D. O. Jacobs, and D. W. Wilmore. Glutathione deficiency increases organ dysfunction after hemorrhagic shock. Surgery 1992, 112:140-7; discussion 8-9.
20. Sacks G., I. Sargent, and C. Redman. An innate view of human pregnancy. Immunol. Today 1999, 20(3): 114-8.
21. Muyan M. and I. Boime I. Secretion of chorionic gonadotropin from human trophoblast. Placenta 1997, 19(4):237-41.
22. Alexander H., G. Zimmermann, M. Lehmann, R. Pfeiffer, E. Schone, S. Leiblein, and M. Ziegert. HCG secretion by peripheral mononuclear cells during pregnancy. Domest. Anim. Endocrinol. 1998, 15(5):377-87.
23. Khan N. A., A. Khan, H. F. Savelkoul, and R. Benner. Inhibition of diabetes in NOD mice by human pregnancy factor. Hum. Immunol. 2001, 62(12): 1315-23.
24. Cole L. A. The deactivation of hCG by nicking and dissociation. J. Clin. Endocrinical. Metab. 1993, 76(3)704-10.
25. Benner R. and N. A. Khan. Dissection of systems, cell populations and molecules. Scand. J. Immunol. 2005, 62 Suppl 1:62-6.
26. Khan N. A., A. Khan, H. F. Savelkoul, and R. Benner. Inhibition of septic shock in mice by an oligopeptide from the beta-chain of human chorionic gonadotropin hormone. Hum. Immunol. 2002, 63:1-7.
27. Beillard E., N. Pallisgaard, V. H. van der Velden, W. Bi, R. Dee, E. van der Schoot, E. Delabesse, E. Macintyre, E. Gottardi, G. Saglio, F. Watzinger, T. Lion, J. J. van Dongen, P. Hokland, and J. Gabert. Evaluation of candidate control genes for diagnosis and residual disease detection in leukemic patients using “real-time” quantitative reverse-transcriptase polymerase chain reaction (RQ-PCR)—a Europe against cancer program. Leukemia 2003, 17:2474-2486.
28. Childs E. W., J. G. Wood, D. M. Smalley, F. A. Hunter, and L. Y. Cheung. Leukocyte adherence and sequestration following hemorrhagic shock and total ischemia in rats. Shock 1999, 11(4):248-52.
29. Suenaert P., V. Bulteel, L. Lemmens, M. Noman, B. Geypens, G. Van Assche, K. Geboes, J. L. Ceuppens, and P. Rutgeerts. Anti-tumor necrosis factor treatment restores the gut barrier in Crohn's disease. Am. J. Gastroenterol. 2002, 97(8):2000-4.
30. Olsen N. J. and C. M. Stein. New drugs for rheumatoid arthritis. N. Engl. J. Med. 2004, 350(21):2167-79.
31. Kox W. J., T. Volk, S. N. Kox, and H. D. Volk. Immunomodulatory therapies in sepsis. Intensive Care Med. 2000, 26 (1)5124-8.
32. Jit M., B. Henderson, M. Stevens, and R. M. Seymour. TNF-alpha neutralization in cytokine-driven diseases: a mathematical model to account for therapeutic success in rheumatoid arthritis but therapeutic failure in systemic inflammatory response syndrome. Rheumatology 2005 March, 44(3):323-31.
33. Yang R., X. Han, T. Uchiyama, S. K. Watkins, A. Yaguchi, R. L. Delude, and M. P. Fink. IL-6 is essential for development of gut barrier dysfunction after hemorrhagic shock and resuscitation in mice. Am. J. Physiol. Gastrointest. Liver Physiol. 2003, 285(3):G621-9.
34. Taniguchi T., Y. Koido, J. Aiboshi, T. Yamashita, S. Suzaki, and A. Kurokawa. Change in the ratio of interleukin-6 to interleukin-10 predicts a poor outcome in patients with systemic inflammatory response syndrome. Crit. Care Med. 1999, 27:1262-1264.
35. Stites D. P., C. S. Pavia, L. E. Clemens, R. W. Kuhn, and P. K. Siiteri. Immunologic regulation in pregnancy. Arthritis Rheum. 1979, 22(11): 1300-7.
36. Saito S. Cytokine network at the feto-maternal interface. J. Reprod. Immunol. 2000, 47(2):87-103.
37. Puett D., Y. Li, G. DeMars, K. Angelova, and F. Fanelli. A functional transmembrane complex: the luteinizing hormone receptor with bound ligand and G protein. Mol. Cell. Endocrinol. 2007, 2, 260-262:126-36.
38. Bai J. P. F., P. Subramanian, H. I. Mosberg, and G. L. Amidon. Structural requirements for the intestinal mucosal-cell peptide transport: the need for N-terminal α-amino group. Pharm. Res. 1991, 8:593-599.
39. Yang R., X. Tan, A. M. Thomas, R. Steppacher, N. Qureshi, D. C. Morrison, and C. W. Van Way 3rd. Alanine-glutamine dipeptide (AGD) inhibits expression of inflammation-related genes in hemorrhagic shock. J. Parenter. Enteral. Nutr. 2007, 31(1):32-6.
Ares M. P., M. Stollenwerk, A. Olsson, B. Kallin, S. Jovinge, and J. Nilsson. Decreased inducibility of TNF expression in lipid-loaded macrophages. BMC Immunol. 2002; 3:13.
Autilio L. A., W. T. Norton, and R. D. Terry. The Preparation and Some Properties of Purified Myelin from the Central Nervous System. J. Neurochem. 1964; 11:17-27.
Becher B., A. Prat, J. P. Antel. Brain-immune connection: immuno-regulatory properties of CNS-resident cells. Glia 2000; 29:293-304.
Cannella B. and C. S. Raine. Multiple sclerosis: Cytokine receptors on oligodendrocytes predict innate regulation. Ann. Neurol. 2004; 55:46-57.
Chayen J. and L. Bitensky. Analysis of chemical components of cells and tissues; reactions for lipids. Practical Histochemistry. West Sussex: Wiley, 1991; 45.
Goerdt S., O. Politz, K. Schledzewski, R. Birk, A. Gratchev, P. Guillot, et al. Alternative versus classical activation of macrophages. Pathobiology 1999; 67:222-6.
Gordon S. Alternative activation of macrophages. Nat. Rev. Immunol. 2003; 3:23-35.
Gratchev A., K. Schledzewski, P. Guillot, and S. Goerdt. Alternatively activated antigen-presenting cells: molecular repertoire, immune regulation, and healing. Skin Pharmacol. Appl. Skin Physiol. 2001; 14:272-9.
Greaves D. R. and S. Gordon. Thematic review series: The Immune System and Atherogenesis. Recent insights into the biology of macrophage scavenger receptors. J. Lipid Res. 2005; 46:11-20.
Greter M., F. L. Heppner, M. P. Lemos, B. M. Odermatt, N. Goebels, T. Laufer, et al. Dendritic cells permit immune invasion of the CNS in an animal model of multiple sclerosis. Nat. Med. 2005; 11:328-34.
Grigoriadis N., T. Ben-Hur, D. Karussis, and I. Milonas. Axonal damage in multiple sclerosis: a complex issue in a complex disease. Clin. Neurol. Neurosurg. 2004; 106:211-7.
Harris S. G., J. Padilla, L. Koumas, D. Ray, and R. P. Phipps. Prostaglandins as modulators of immunity. Trends Immunol. 2002; 23:144-50.
Heppner F. L., M. Greter, D. Marino, J. Falsig, G. Raivich, N. Hovelmeyer, et al. Experimental autoimmune encephalomyelitis repressed by microglial paralysis. Nat. Med. 2005; 11:146-52.
Hinz B., K. Brune, and A. Pahl. Prostaglandin E(2) up-regulates cyclooxygenase-2 expression in lipopolysaccharide-stimulated RAW 264.7 macrophages. Biochem. Biophys. Res. Commun. 2000; 272:744-8.
Hoefakker S., W. J. Boersma, and E. Claassen. Detection of human cytokines in situ using antibody and probe based methods. J. Immunol. Methods 1995; 185:149-75.
Ikegami R., Y. Sugimoto, E. Segi, M. Katsuyama, H. Karahashi, F. Amano, et al. The expression of prostaglandin E receptors EP2 and EP4 and their different regulation by lipopolysaccharide in C3H/HeN peritoneal macrophages. J. Immunol. 2001; 166:4689-96.
Joseph S. B., M. N. Bradley, A. Castrillo, K. W. Bruhn, P. A. Mak, L. Pei, et al. LXR-dependent gene expression is important for macrophage survival and the innate immune response. Cell 2004; 119:299-309.
Joseph S. B., A. Castrillo, B. A. Laffitte, D. J. Mangelsdorf, and P. Tontonoz. Reciprocal regulation of inflammation and lipid metabolism by liver X receptors. Nat. Med. 2003; 9:213-9.
Kalinski P., C. M. Hilkens, A. Snijders, F. G. Snijdewint, and M. L. Kapsenberg. Dendritic cells, obtained from peripheral blood precursors in the presence of PGE2, promote Th2 responses. Adv. Exp. Med. Biol. 1997; 417:363-7.
Kodelja V., C. Muller, O. Politz, N. Hakij, C. E. Orfanos, and S. Goerdt. Alternative macrophage activation-associated CC-chemokine-1, a novel structural homologue of macrophage inflammatory protein-1 alpha with a Th2-associated expression pattern. J. Immunol. 1998; 160:1411-8.
Kuhlmann T., G. Lingfeld, A. Bitsch, J. Schuchardt, and W. Bruck. Acute axonal damage in multiple sclerosis is most extensive in early disease stages and decreases over time. Brain 2002; 125:2202-12.
Lassmann H. Recent neuropathological findings in MS—implications for diagnosis and therapy. J. Neurol. 2004; 251 Suppl 4:IV2-5.
Lawrence T., D. A. Willoughby, and D. W. Gilroy. Anti-inflammatory lipid mediators and insights into the resolution of inflammation. Nat. Rev. Immunol. 2002; 2:787-95.
Li H., M. L. Cuzner, and J. Newcombe. Microglia-derived macrophages in early multiple sclerosis plaques. Neuropathol. Appl. Neurobiol. 1996; 22:207-15.
Lo C. J., M. Fu, F. R. Lo, and H. G. Cryer. Macrophage TNF mRNA expression induced by LPS is regulated by sphingomyelin metabolites. Shock 1999; 11:411-5.
Mantovani A., A. Sica, S. Sozzani, P. Allavena, A. Vecchi, and M. Locati. The chemokine system in diverse forms of macrophage activation and polarization. Trends Immunol. 2004; 25:677-86.
Mantovani A., S. Sozzani, M. Locati, P. Allavena, and A. Sica. Macrophage polarization: tumor-associated macrophages as a paradigm for polarized M2 mononuclear phagocytes. Trends Immunol. 2002; 23:549-55.
Matute C. and F. Perez-Cerda. Multiple sclerosis: novel perspectives on newly forming lesions. Trends Neurosci. 2005; 28:173-5.
McMahon E. J., S. L. Bailey, C. V. Castenada, H. Waldner, and S. D. Miller. Epitope spreading initiates in the CNS in two mouse models of multiple sclerosis. Nat. Med. 2005; 11:335-339.
Mosser D. M. The many faces of macrophage activation. J. Leukoc. Biol. 2003; 73:209-12.
Newcombe J., H. Li, and M. L. Cuzner. Low density lipoprotein uptake by macrophages in multiple sclerosis plaques: implications for pathogenesis. Neuropathol. Appl. Neurobiol. 1994; 20:152-62.
Norton W. T. and S. E. Poduslo. Myelination in rat brain: method of myelin isolation. J. Neurochem. 1973; 21:749-57.
Pettus B. J., C. E. Chalfant, and Y. A. Hannun. Ceramide in apoptosis: an overview and current perspectives. Biochim. Biophys. Acta. 2002; 1585:114-25.
Platten M. and L. Steinman. Multiple sclerosis: trapped in deadly glue. Nat. Med. 2005; 11:252-3.
Raine C. S. Multiple sclerosis: immune system molecule expression in the central nervous system. J. Neuropathol. Exp. Neurol. 1994; 53:328-37.
Sospedra M. and R. Martin. Immunology of multiple sclerosis *. Annu. Rev. Immunol. 2005; 23:683-747.
van der Fits L., L. I. van der Wel, J. D. Laman, E. P. Prens, and M. C. Verschuren. Psoriatic lesional skin exhibits an aberrant expression pattern of interferon regulatory factor-2 (IRF-2). J. Pathol. 2003; 199:107-14.
van der Laan L. J., S. R. Ruuls, K. S. Weber, I. J. Lodder, E. A. Dopp, and C. D. Dijkstra. Macrophage phagocytosis of myelin in vitro determined by flow cytometry: phagocytosis is mediated by CR3 and induces production of tumor necrosis factor-alpha and nitric oxide. J. Neuroimmunol. 1996; 70:145-52.
Varadhachary A. S., M. Monestier, and P. Salgame. Reciprocal induction of IL-10 and IL-12 from macrophages by low-density lipoprotein and its oxidized forms. Cell. Immunol. 2001; 213:45-51.
Vulcano M., S. Struyf, P. Scapini, M. Cassatella, S. Bernasconi, R. Bonecchi, et al. Unique regulation of CCL18 production by maturing dendritic cells. J. Immunol. 2003; 170:3843-9.
Wingerchuk D. M., C. F. Lucchinetti, and J. H. Noseworthy. Multiple sclerosis: current pathophysiological concepts. Lab. Invest. 2001; 81:263-81.
Woelk H. and P. Borri. Lipid and fatty acid composition of myelin purified from normal and MS brains. Eur. Neurol. 1973; 10:250-60.

### FURTHER REFERENCES

WO 99/59671,
WO 01/72831,
WO 97/49721,
WO 01/10907,
WO 01/11048.

The contents of the entirety of each reference identified herein is incorporated in its entirety by this reference.

What is claimed is:

1. A method for treating a subject suspected to suffer from a disease comprising an inflammatory condition, the method comprising:
subjecting the subject to a diagnostic process aimed at determining inflammatory disease stage of the subject;
providing the subject with a gene-regulatory peptide or functional analogue thereof depending on the outcome of the determination of disease stage wherein the subject is provided with a peptide selected from the group consisting of LQGV (SEQ ID NO:1), MTR, MTRV (SEQ ID NO:20), AQGV (SEQ ID NO:2), LAGV (SEQ ID NO:10), AQG, LQG, VLPALPQ (SEQ ID NO:13), LAG, and VLPALP (SEQ ID NO:4).

2. The method according to claim 1, wherein the peptide is selected from the group consisting of LQGV (SEQ ID NO:1), MTR, MTRV (SEQ ID NO:20), AQGV (SEQ ID NO:2), LAGV (SEQ ID NO:10), AQG, and LQG.

3. The method according to claim 2, wherein the peptide is selected from the group consisting of LQGV (SEQ ID NO:1), LAGV (SEQ ID NO:10), and AQGV.

4. A method according to claim 2, wherein the peptide is selected from the group consisting of LQGV (SEQ ID NO:1), MTR, and MTRV (SEQ ID NO:20).


SEQUENCE LISTING (29 sequences)

SEQ ID NO: 1 - protein, 4 aa, Artificial Sequence
  Description of Artificial Sequence Synthetic peptide
LQGV

SEQ ID NO: 2 - protein, 4 aa, Artificial Sequence
  Description of Artificial Sequence Synthetic peptide
AQGV

SEQ ID NO: 3 - protein, 4 aa, Artificial Sequence
  Description of Artificial Sequence Synthetic peptide
LQGA

SEQ ID NO: 4 - protein, 6 aa, Artificial Sequence
  Description of Artificial Sequence Synthetic peptide
VLPALP

SEQ ID NO: 5 - protein, 6 aa, Artificial Sequence
  Description of Artificial Sequence Synthetic peptide
ALPALP

SEQ ID NO: 6 - protein, 6 aa, Artificial Sequence
  Description of Artificial Sequence Synthetic peptide
VAPALP

SEQ ID NO: 7 - protein, 7 aa, Artificial Sequence
  Description of Artificial Sequence Synthetic peptide
ALPALPQ

SEQ ID NO: 8 - protein, 7 aa, Artificial Sequence
  Description of Artificial Sequence Synthetic peptide
VLPAAPQ

SEQ ID NO: 9 - protein, 7 aa, Artificial Sequence
  Description of Artificial Sequence Synthetic peptide
VLPALAQ

SEQ ID NO: 10 - protein, 4 aa, Artificial Sequence
  Description of Artificial Sequence Synthetic peptide
LAGV

SEQ ID NO: 11 - protein, 6 aa, Artificial Sequence
  Description of Artificial Sequence Synthetic peptide
VLAALP

SEQ ID NO: 12 - protein, 6 aa, Artificial Sequence
  Description of Artificial Sequence Synthetic peptide
VLPALA

SEQ ID NO: 13 - protein, 7 aa, Artificial Sequence
  Description of Artificial Sequence Synthetic peptide
VLPALPQ

SEQ ID NO: 14 - protein, 7 aa, Artificial Sequence
  Description of Artificial Sequence Synthetic peptide
VLAALPQ

SEQ ID NO: 15 - protein, 7 aa, Artificial Sequence
  Description of Artificial Sequence Synthetic peptide
VLPALPA

SEQ ID NO: 16 - protein, 7 aa, Artificial Sequence
  Description of Artificial Sequence Synthetic peptide
GVLPALP

SEQ ID NO: 17 - protein, 13 aa, Artificial Sequence
  Description of Artificial Sequence Synthetic peptide
LQGVLPALPQVVC

SEQ ID NO: 18 - protein, 13 aa, Artificial Sequence
  Description of Artificial Sequence Synthetic peptide
LPGCPRGVNPVVS

SEQ ID NO: 19 - protein, 4 aa, Artificial Sequence
  Description of Artificial Sequence Synthetic peptide
LPGC

SEQ ID NO: 20 - protein, 4 aa, Artificial Sequence
  Description of Artificial Sequence Synthetic peptide
MTRV

SEQ ID NO: 21 - protein, 10 aa, Artificial Sequence
  Description of Artificial Sequence Synthetic peptide
VLPALPQVVC

SEQ ID NO: 22 - protein, 10 aa, Artificial Sequence
  Description of Artificial Sequence Synthetic peptide
LQGVLPALPQ

SEQ ID NO: 23 - protein, 8 aa, Artificial Sequence
  Description of Artificial Sequence Synthetic peptide
GVLPALPQ

SEQ ID NO: 24 - protein, 38 aa, Artificial Sequence
  Description of Artificial Sequence Synthetic peptide
VVCNYRDVRFESIRLPGCPRGVNPVVSYAVALSCQCAL

SEQ ID NO: 25 - protein, 35 aa, Artificial Sequence
  Description of Artificial Sequence Synthetic peptide
RPRCRPINATLAVEKEGCPVCITVNTTICAGYCPT

SEQ ID NO: 26 - protein, 18 aa, Artificial Sequence
  Description of Artificial Sequence Synthetic peptide
SKAPPPSLPSPSRLPGPS

SEQ ID NO: 27 - protein, 16 aa, Artificial Sequence
  Description of Artificial Sequence Synthetic peptide
SIRLPGCPRGVNPVVS

SEQ ID NO: 28 - DNA, 25 bp, Artificial Sequence
  Description of Artificial Sequence Synthetic probe
agctcagagggggactttccgagag

SEQ ID NO: 29 - protein, 4 aa, Artificial Sequence
  Description of Artificial Sequence Synthetic peptide
QVVC